(19) United States
(12) Patent Application Publication
(10) Pub. No.: US 2026/0250411 A1
(43) Pub. Date: Aug. 27, 2026

(54) ANTI-PSGL-1 ANTIBODIES AND USES THEREOF

(71) Applicant: AltruBio Inc., Wilmington, DE (US)

(72) Inventors: Gene Woo LEE, Chelmsford, MA (US); Shih-Yao LIN, Taipei (TW); Feng-Lin CHIANG, Taipei (TW)

(73) Assignee: AltruBio Inc., Wilmington, DE (US)

(21) Appl. No.: 19/664,758

(22) Filed: Apr. 30, 2026

Related U.S. Application Data
(63) Continuation of application No. PCT/US2024/056078, filed on Nov. 15, 2024.
(60) Provisional application No. 63/600,598, filed on Nov. 17, 2023.

(51) Int. Cl. C07K 16/28; A61K 31/519; A61P 35/02

(52) U.S. Cl. CPC C07K 16/2896; A61K 31/519; A61P 35/02; C07K 2317/24; C07K 2317/31; C07K 2317/35; C07K 2317/565; C07K 2317/73; C07K 2317/734; C07K 2317/92

(57) ABSTRACT

Provided herein are antibodies, such as IgM antibodies, that bind to human PSGL-1. These antibodies can be used in a variety of diagnostic and therapeutic methods, including, without limitation, treating T-cell mediated inflammatory diseases, cancers, transplantations, and transfusions.

### CROSS-REFERENCE TO RELATED APPLICATIONS

This application is a continuation of International Application No. PCT/US2024/056078, filed Nov. 15, 2024, which claims the priority benefit of U.S. Provisional Application No. 63/600,598, filed Nov. 17, 2023, each of which is incorporated herein by reference in its entirety.

### REFERENCE TO AN ELECTRONIC SEQUENCE LISTING

The content of the electronic sequence listing (60/659,2001901SeqList.xml; Size: 24,910 bytes; and Date of Creation: Apr. 29, 2026) is incorporated herein by reference in its entirety.

### FIELD

Provided herein are IgM antibodies that bind to human P-selectin glycoprotein ligand-1 (PSGL-1), as well as polynucleotides, vectors, host cells, methods, pharmaceutical compositions, kits, and uses related thereto. These antibodies may find use in a variety of diagnostic and therapeutic methods, including, without limitation, treating T-cell mediated inflammatory diseases, transplantations, transfusions, and cancer.

### BACKGROUND

Inflammatory responses to infection or injury are initiated by the adherence of leukocytes to the vascular wall (McEver et al, 1997, J. Clin. Invest., 100 (3): 485-492). Selectin represents a family of glycoproteins which mediate the first leukocyte-endothelial cell and leukocyte-platelet interactions during inflammation. The selectin family, which consists of L-selectin, E-selectin, and P-selectin, comprises an NH₂-terminal lectin domain, followed by an EGF-like domain, a series of consensus repeats, a transmembrane domain, and a short cytoplasmic tail. The lectin domains of selectins interact with specific glycoconjugate ligands in order to facilitate cell adhesion. L-selectin, expressed on most leukocytes, binds to ligands on some endothelial cells and other leukocytes. E-selectin, expressed on cytokine activated endothelial cells, binds to ligands on most leukocytes. P-selectin, expressed on activated platelets and endothelial cells, also binds to ligands on most leukocytes.

P-selectin glycoprotein ligand-1 (“PSGL-1”), also known as SELPLG or CD162 (cluster of differentiation 162) is a human mucin-type glycoprotein ligand for all three selectins (Constantin, Gabriela, 2004, Drug News Perspect., 17(9): 579-585; McEver et al., 1997, J. Clin. Invest., 100 (3): 485-492). PSGL-1 is a disulfide-bonded homodimer with two 120-kD subunits, and is expressed on the surface of monocytes, lymphocytes, granulocytes, and in some CD34⁺ stem cells. PSGL-1 is likely to contribute to pathological leukocyte recruitment in many inflammatory disorders since it facilitates the adhesive interactions of selectins. In addition, PSGL-1 is shown to have a unique regulatory role in T-cells. Mice deficient in PSGL-1 show enhanced proliferative responses and autoimmunity, suggesting that PSGL-1 plays an important role in down-regulating T-cell responses (Krystle M. et al. J. Immunol. 2012; 188:1638-1646; Urzainqui et al. Ann Rheum Dis 2013; 71:650; Pdrez-Frias A, et al. Arthritis Rheumatol. 2014 November; 66(11):3178-89; Angiari et al. J Immunol. 2013; 191(11):5489-500).

Several anti-PSGL-1 antibodies have been developed (see, e.g., International Application Pub. Nos. WO 2005/110475, WO 2003/013603, and WO 2012/174001; Constantin, Gabriela, 2004, Drug News Perspect., 17(9): 579-585; Chen et al. Blood. 2004; 104(10):3233-42; Huang et al, Eur J Immunol. 2005; 35(7):2239-49; and U.S. Pat. No. 7,604,800). Some existing anti-PSGL-1 antibodies preferentially induce apoptosis of late-stage activated T-cells but not other PSGL-1-expressing cells, and may be useful as anti-inflammatory or anti-cancer therapeutics, or for use in transplantations and/or transfusions.

However, despite the considerable success of monoclonal antibodies as therapeutics, the therapeutic outcomes are sometimes less than satisfactory (Scott et al., Antibody Therapy of Cancer. Nat. Rev. Cancer 2012, 12, 278-287). Although many antibodies agonize specific cell-surface receptors, monovalent or divalent binding does not always elicit sufficient signaling of downstream pathways (Lian et al., Broadening and Enhancing Functions of Antibodies by Self-Assembling Multimerization at Cell Surface. ACS Nano, 2019, 13: 11422-11432). In many cases, downstream signaling also requires antibody-binding receptors to assemble into oligomeric complexes (Kiessling et al., Synthetic Multivalent Ligands as Probes of Signal Transduction. Angew. Chem., Int. Ed. 2006, 45, 2348-2368; Zhang et al., DNA-Scaffolded Multivalent Ligands to Modulate Cell Function. ChemBioChem 2014, 15, 1268-1273). In fact, ligand-induced receptor clustering is critical for magnifying apoptosis (Li et al., Amplification of CD20 Cross-Linking in Rituximab-Resistant B-Lymphoma Cells Enhances Apoptosis Induction by Drug-Free Macromolecular Therapeutics. ACS Nano 2018, 12, 3658-3670; Aluri et al., A Hybrid Protein-Polymer Nanoworm Potentiates Apoptosis Better Than a Monoclonal Antibody. ACS Nano 2014, 8, 2064-2076), and can be driven by multivalent constructs of antibodies attached to nanoparticles or polymers (Riley et al., Frizzled7 Antibody-Functionalized Nanoshells Enable Multivalent Binding for Wnt Signaling Inhibition in Triple Negative Breast Cancer Cells. Small 2017, 13, 1700544; Zhang et al., Generation of Rituximab Polymer May Cause Hyper-Cross-Linking-Induced Apoptosis in Non-Hodgkin's Lymphomas. Clin. Cancer Res. 2005, 11, 5971-5980; Li et al., Synergistic Anti-Tumor Therapy by a Comb-Like Multifunctional Antibody Nanoarray with Exceptionally Potent Activity. Sci. Rep. 2015, 5, 15712).

Antibodies of the IgM class are multimeric (e.g., pentamers or hexamers), and are particularly well-suited to binding and multimerization of target antigens (see, e.g., Keyt et al., Antibodies (Basel). 2020 Oct. 13; 9(4):53). IgM antibodies have been used to multimerize cell surface receptors to modulate downstream signaling.

Accordingly, a need exists for improved anti-PSGL-1 antibodies, such as IgM antibodies, with greater efficacy than existing antibodies.

All publications, patents, and patent applications cited herein are hereby incorporated by reference in their entirety for all purposes.

### BRIEF SUMMARY

To meet this need, provided herein are IgM antibodies that specifically bind to human PSGL-1, as well as polynucleotides, vectors, host cells, methods, pharmaceutical compositions, kits, and uses related thereto. The present disclosure demonstrates that IgM antibodies that specifically bind to human PSGL-1 have greater potency and efficacy than other anti-PSGL-1 antibodies, such as bivalent anti-PSGL-1 antibodies. As such, these IgM antibodies may find use, inter alia, in diagnostic and/or therapeutic methods, uses, and compositions related to T-cell function, such as in treating T-cell mediated inflammatory diseases, cancers, transfusions, and/or transplantations.

In certain aspects, provided herein is an IgM antibody that binds to a human P-selectin glycoprotein ligand-1 (PSGL-1) protein, wherein the antibody comprises a heavy chain comprising a heavy chain variable domain and a light chain comprising a light chain variable domain, wherein: the light chain variable domain comprises a CDR-L1 comprising the amino acid sequence RSSQSIVHNDGNTYFE (SEQ ID NO: 1), a CDR-L2 comprising the amino acid sequence KVSNRFS (SEQ ID NO: 2), and a CDR-L3 comprising the amino acid sequence FQGSYVPLT (SEQ ID NO: 3); and the heavy chain variable domain comprises a CDR-H1 comprising the amino acid sequence SFGMH (SEQ ID NO: 4), a CDR-H2 comprising the amino acid sequence YINGGSSTIFYANAVKG (SEQ ID NO: 5), and a CDR-H3 comprising the amino acid sequence YASYGGGAMDY (SEQ ID NO: 6).

In some embodiments, the human PSGL-1 protein comprises the amino acid sequence of SEQ ID NO: 21 or SEQ ID NO: 22.

In some embodiments, the antibody is a chimeric, humanized or human antibody. In some embodiments, the antibody is a bispecific or multi-specific antibody.

In some embodiments, the heavy chain variable domain comprises the amino acid sequence of SEQ ID NO: 16, or an amino acid sequence having at least about any of 80%, 85%, 90%, 95%, or 99% identity thereto. In some embodiments, the light chain variable domain comprises the amino acid sequence of SEQ ID NO: 15, or an amino acid sequence having at least about any of 80%, 85%, 90%, 95%, or 99% identity thereto. In some embodiments, the heavy chain variable domain comprises the amino acid sequence of SEQ ID NO: 16, or an amino acid sequence having at least about any of 80%, 85%, 90%, 95%, or 99% identity thereto, and the light chain variable domain comprises the amino acid sequence of SEQ ID NO: 15, or an amino acid sequence having at least about any of 80%, 85%, 90%, 95%, or 99% identity thereto. In some embodiments, the heavy chain variable domain comprises the amino acid sequence of SEQ ID NO: 16, and the light chain variable domain comprises the amino acid sequence of SEQ ID NO: 15. In some embodiments, the antibody further comprises a human IgM heavy chain constant region (e.g., comprising the amino acid sequence of SEQ ID NO: 18).

In some embodiments, the antibody is a multivalent antibody. In some embodiments, the antibody is a pentameric IgM antibody. In some embodiments, the antibody is a hexameric IgM antibody.

In some embodiments, the antibody comprises a J-chain, or a fragment or variant thereof. In some embodiments, the J-chain comprises the amino acid sequence of SEQ ID NO: 19, or an amino acid sequence having at least about any of 80%, 85%, 90%, 95%, or 99% identity thereto.

In some embodiments, the heavy chain comprises an IgM heavy chain constant region, or a fragment or variant thereof, optionally wherein the IgM heavy chain constant region is a human IgM heavy chain constant region or a fragment or variant thereof. In some embodiments, the IgM heavy chain constant region comprises a Cμ1 domain, a Cμ2 domain, a Cμ3 domain, and/or a Cμ4 domain.

In some embodiments, the antibody has reduced complement-dependent cytotoxicity (CDC) activity as compared to a corresponding wild-type IgM antibody. In some embodiments, the antibody comprises one or more amino acid substitutions in the IgM constant region that confer reduced CDC activity as compared to the corresponding wild-type IgM antibody.

In some embodiments, the IgM heavy chain constant region comprises a tailpiece polypeptide C-terminal to the Cμ4 domain. In some embodiments, the IgM heavy chain constant region comprises the Cμ3 domain, the Cμ4 domain, and the tailpiece polypeptide C-terminal to the Cμ4 domain. In some embodiments, the antibody further comprises the Cμ1 domain and/or the Cμ2 domain.

In some embodiments, the heavy chain comprises an IgM heavy chain constant region comprising: (a) the amino acid sequence of SEQ ID NO: 18, or an amino acid sequence with at least about any of 80%, 85%, 90%, 95%, or 99% identity thereto.

In some embodiments, the light chain is a human kappa light chain. In some embodiments, the light chain comprises a human kappa light chain constant region. In some embodiments, the light chain comprises the amino acid sequence of SEQ ID NO: 17, or an amino acid sequence with at least about any of 80%, 85%, 90%, 95%, or 99% identity thereto. In some embodiments, the heavy chain comprises: (a) the amino acid sequence of SEQ ID NO: 20, or an amino acid sequence with at least about any of 80%, 85%, 90%, 95%, or 99% identity thereto. In some embodiments, the light chain comprises the amino acid sequence of SEQ ID NO: 17, or an amino acid sequence with at least about any of 80%, 85%, 90%, 95%, or 99% identity thereto, and the heavy chain comprises the amino acid sequence of SEQ ID NO: 20, or an amino acid sequence with at least about any of 80%, 85%, 90%, 95%, or 99% identity thereto. In some embodiments, the light chain comprises the amino acid sequence of SEQ ID NO: 17, and the heavy chain comprises the amino acid sequence of SEQ ID NO: 20.

In some embodiments, the antibody induces apoptosis of activated T cells to a greater extent than a control anti-PSGL-1 antibody or antibody fragment, e.g., a bivalent anti-PSGL-1 antibody or antibody fragment. In some embodiments, the control anti-PSGL-1 antibody comprises a heavy chain variable domain and a light chain variable domain, wherein the light chain variable domain comprises a CDR-L1 comprising the amino acid sequence RSSQSIVHNDGNTYFE (SEQ ID NO: 1), a CDR-L2 comprising the amino acid sequence KVSNRFS (SEQ ID NO: 2), and a CDR-L3 comprising the amino acid sequence FQGSYVPLT (SEQ ID NO: 3); and the heavy chain variable domain comprises a CDR-H1 comprising the amino acid sequence SFGMH (SEQ ID NO: 4), a CDR-H2 comprising the amino acid sequence YINGGSSTIFYANAVKG (SEQ ID NO: 5), and a CDR-H3 comprising the amino acid sequence YASYGGGAMDY (SEQ ID NO: 6). In some embodiments, the control anti-PSGL-1 antibody comprises a heavy chain variable domain comprising the amino acid sequence of SEQ ID NO: 16 and a light chain variable domain comprising the amino acid sequence of SEQ ID NO: 15. In some embodiments, the control anti-PSGL-1 antibody is not an IgM antibody. In some embodiments, the control anti-PSGL-1 antibody is an IgG antibody. In some embodiments, the control anti-PSGL-1 antibody comprises a light chain comprising the amino acid sequence of SEQ ID NO:23 and a heavy chain comprising the amino acid sequence of SEQ ID NO:24. In some embodiments, one or two Fc domains of the control antibody do not have the C-terminal lysine residue.

In another aspect, provided herein are one or more isolated nucleic acids encoding the antibody of any one of the above embodiments. In another aspect, provided herein is a vector comprising the nucleic acid of any one of the above embodiments. In another aspect, provided herein is a host cell comprising the nucleic acid or the vector of any one of the above embodiments. In another aspect, provided herein is a method of producing an anti-PSGL-1 antibody, comprising culturing a host cell of any one of the above embodiments so that the antibody is produced. In some embodiments, the method further comprises recovering the antibody from the host cell. In other aspects, further provided herein are anti-PSGL-1 antibodies produced by the methods of producing an anti-PSGL-1 antibody of any one of the above embodiments. In another aspect, provided herein is a pharmaceutical composition comprising the antibody of any one of the above embodiments and a pharmaceutically acceptable carrier.

In another aspect, provided herein is a kit comprising the antibody of any one of the above embodiments and an optional pharmaceutically acceptable carrier. In some embodiments, the kit further comprises a package insert comprising instructions for administration of the antibody to treat a T-cell mediated inflammatory disease in a subject in need thereof. In some embodiments, the kit further comprises a package insert comprising instructions for administration of the antibody before, concurrently with, and/or after a transfusion or transplantation in a subject in need thereof. In some embodiments, the kit further comprises a package insert comprising instructions for administration of the antibody to treat a cancer in a subject in need thereof. In some embodiments, the package insert further comprises instructions for using the antibody in combination with a Janus kinase (JAK) inhibitor. In some embodiments, the kit further comprises a JAK inhibitor.

In another aspect, provided herein is a method of treating a T-cell mediated inflammatory disease, the method comprising administering to a subject in need thereof a therapeutically effective amount of the antibody of any one of the above embodiments. In another aspect, provided herein is a method of treating a subject in need of a transfusion or transplantation, comprising administering to a subject in need thereof a therapeutically effective amount of the antibody of any one of the above embodiments before, concurrently with, and/or after a transfusion or transplantation. In another aspect, provided herein is a method of treating a cancer, the method comprising administering to a subject in need thereof a therapeutically effective amount of the antibody of any one of the above embodiments. In some embodiments, the method further comprises administering a JAK inhibitor to the subject.

In another aspect, provided herein is an antibody of any one of the above embodiments for use in treating a T-cell mediated inflammatory disease in a subject in need thereof. In another aspect, provided herein is an antibody of any one of the above embodiments for use in treating a subject in need of a transfusion or transplantation. In another aspect, provided herein is an antibody of any one of the above embodiments for use in treating a cancer in a subject in need thereof. In some embodiments, the antibody is for use in combination with a JAK inhibitor.

In another aspect, provided herein is a use of the antibody of any one of the above embodiments in the manufacture of a medicament for treating a T-cell mediated inflammatory disease in a subject in need thereof. In another aspect, provided herein is a use of the antibody of any one of the above embodiments in the manufacture of a medicament for treating a subject in need of a transfusion or transplantation. In another aspect, provided herein is a use of the antibody of any one of the above embodiments in the manufacture of a medicament for treating a cancer in a subject in need thereof. In some embodiments, the antibody is to be administered in combination with a JAK inhibitor.

In some embodiments, which may be combined with any of the preceding aspects or embodiments, the T-cell mediated inflammatory disease is an autoimmune disease. In some embodiments, which may be combined with any of the preceding aspects or embodiments, the T-cell mediated inflammatory disease is selected from the group consisting of: psoriasis, psoriatic arthritis, rheumatoid arthritis, Crohn's disease, ankylosing spondylitis, type I diabetes, ulcerative colitis, multiple sclerosis, allergy, atopic dermatitis, asthma, graft versus host disease (GVHD), vitiligo, alopecia areata, Steven Johnson Syndrome (SJS), Drug reaction with eosinophilia and systemic symptoms (Dress), T-cell mediated skin diseases, juvenile arthritis, lupus, inflammatory bowel disease, myasthenia gravis, immunoglobulin nephropathies, myocarditis, and autoimmune thyroid disorder. In some embodiments, which may be combined with any of the preceding aspects or embodiments, the GVHD is acute GVHD, steroid-refractory acute GVHD (SR-aGVHD), treatment-refractory acute GVHD (TR-aGVHD), or chronic GVHD. In some embodiments, which may be combined with any of the preceding aspects or embodiments, the transplantation is a transplantation of a tissue selected from the group consisting of bone marrow, kidney, heart, liver, neuronal tissue, lung, pancreas, skin, and intestine. In some embodiments, which may be combined with any of the preceding aspects or embodiments, the transfusion is a transfusion comprising one or more of white blood cells, red blood cells, and platelets. In some embodiments, which may be combined with any of the preceding aspects or embodiments, the cancer is a T-cell neoplasm, optionally a T-cell leukemia or T-cell lymphoma. In some embodiments, which may be combined with any of the preceding aspects or embodiments, the T-cell leukemia or lymphoma is adult T-cell leukemia/lymphoma (ATLL) or cutaneous T-cell lymphoma (CTCL). In some embodiments, which may be combined with any of the preceding aspects or embodiments, the subject is a human. In some embodiments, which may be combined with any of the preceding aspects or embodiments, the JAK inhibitor inhibits JAK1 and/or JAK2. In some embodiments, which may be combined with any of the preceding aspects or embodiments, the JAK inhibitor inhibits JAK1 and/or JAK3. In some embodiments, which may be combined with any of the preceding aspects or embodiments, the JAK inhibitor is ruxolitinib. In some embodiments, which may be combined with any of the preceding aspects or embodiments, the JAK inhibitor is tofacitinib.

It is to be understood that one, some, or all of the properties of the various embodiments described herein may be combined to form other embodiments of the present invention. These and other aspects of the invention will become apparent to one of skill in the art.

### BRIEF DESCRIPTION OF THE DRAWINGS

FIGS. 1A-1B show the functional activity of anti-hPSGL-1 mAb, as measured by percentage of apoptotic cells (assayed by Annexin V+propidium iodide (PI) staining), in inducing apoptosis of SP2/O cells expressing hPSGL-1 (FIG. 1A) or activated primary T cells (FIG. 1B). Shown are percentage of apoptotic cells vs. mAb concentration (μg/mL, as indicated).

FIGS. 2A-2B show characterization of 15A7H-IgM antibody. FIG. 2A shows analysis by SDS-PAGE. FIG. 2B shows analysis by HPLC-SEC.

FIGS. 3A-3C show detection of anti-hPSGL-1 mAb binding activity with recombinant PSGL-1 by ELISA. FIG. 3A shows the binding of 15A7H-IgG4 or 15A7H-IgM to recombinant PSGL-1 with antigen coating at high density (0.5 μg/mL), including corresponding EC50 values (pM) for each (141 pM for 15A7H-IgG4 and 92.6 pM for 15A7H-IgM). FIG. 3B shows the binding of 15A7H-IgG4 or 15A7H-IgM to recombinant PSGL-1 with antigen coating at lower density (0.05 pg/mL), including corresponding EC50 values (pM) for each (97.4 pM for 15A7H-IgG4 and 45.9 pM for 15A7H-IgM).

FIG. 3C shows the binding of 15A7H-IgG4 or 15A7H-IgM to recombinant PSGL-1 with antigen coating at lower density (0.03 pg/mL), including corresponding EC50 values (pM) for each (120 pM for 15A7H-IgG4 and 39.6 pM for 15A7H-IgM).

FIGS. 4A-4C show in vitro functional activities of 15A7H-IgG4, 15A7H-IgG4 in the presence of cross-linker, and 15A7H-IgM, as measured by apoptotic cells (Annexin V+PI staining, detected by flow cytometry). Shown are percentage of apoptotic cells vs. molar concentration of antibody, as indicated (top); percentage of apoptotic cells vs. weight concentration of antibody (pg/mL), as indicated (middle); and EC50 values for each antibody (nM and μg/mL). FIGS. 4A-4C show respective results using PBMCs from 3 different donors.

### DETAILED DESCRIPTION

Provided herein are IgM antibodies that specifically bind to human PSGL-1, as well as nucleic acids, vectors, host cells, methods, pharmaceutical compositions, uses, and kits related thereto. For example, the IgM antibodies of the present disclosure may find use in treating a T-cell mediated inflammatory disease, cancer, or in administration before, concurrently with, and/or after a transfusion or transplantation. The present disclosure is based, at least in part, on the findings disclosed herein that an exemplary anti-PSGL-1 IgM antibody showed more effective binding to hPSGL-1 extracellular domain antigen, as well as superior induction of apoptosis, as compared to a corresponding anti-PSGL-1 IgG antibody. Anti-PSGL-1 IgM antibody induced apoptosis of activated human T cells with far greater potency than anti-PSGL-1 IgG antibody, even when the IgG antibody was cross-linked.

### I. Definitions

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also polypeptides comprising fragments thereof (such as Fab, Fab′, F(ab′)2, Fv); single-chain variable fragments (scFv), single-chain diabodies (scDbs), tandem single-chain variable fragment (scFv) units (termed taFv for tandem scFv), and mutants or other configurations thereof; fusion proteins comprising an antibody portion; and any other modified configuration of the immunoglobulin molecule that comprises an antigen recognition site.

An antibody includes an antibody of any class, such as IgG, IgA, or IgM (or a sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgAQ1, and IgA2. The heavy chain constant domains that correspond to the different classes of immunoglobulins are called alpha, delta, epsilon, gamma, and mu, respectively. The subunit structures and three-dimensional configurations of different classes of immunoglobulins are well known.

The antibodies of the present disclosure are further intended to include bispecific, multispecific, chimeric, humanized, and recombinantly constructed molecules having affinity for a polypeptide conferred by at least one CDR region of the antibody. Single domain antibodies which are either the variable domain of an antibody heavy chain or the variable domain of an antibody light chain are known in the art. See, e.g., Holt et al., Trends Biotechnol. 21:484-490, 2003. Methods of making antibodies comprising either the variable domain of an antibody heavy chain or the variable domain of an antibody light chain, containing three of the six naturally occurring complementarity determining regions from an antibody, are also known in the art. See, e.g., Muyldermans, Rev. Mol. Biotechnol. 74:277-302, 2001.

As used herein, “monoclonal antibody” refers to an antibody of substantially homogeneous antibodies, i.e., the individual antibodies comprising the population are identical except for possible naturally-occurring mutations that may be present in minor amounts. Monoclonal antibodies are generally highly specific, being directed against a single antigenic site. Furthermore, in contrast to polyclonal antibody preparations, which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method first described by Kohler and Milstein, 1975, Nature, 256:495, or may be made by recombinant DNA methods such as described in U.S. Pat. No. 4,816,567. The monoclonal antibodies may also be isolated from phage libraries generated using the techniques described in McCafferty et al., 1990, Nature, 348:552-554, for example.

As used herein, a “chimeric antibody” refers to an antibody having a variable region or part of a variable region from a first species and a constant region from a second species. The production of chimeric antibodies is known in the art (Cabilly et al. (1984), Proc. Natl. Acad. Sci. USA, 81:3273-3277; Harlow and Lane (1988), Antibodies: a Laboratory Manual, Cold Spring Harbor Laboratory). Typically, in these chimeric antibodies, the variable region of both light and heavy chains mimics the variable regions of antibodies derived from one species of mammal, while the constant portions are homologous to the sequences in antibodies derived from another. One clear advantage to such chimeric forms is that, for example, the variable regions can conveniently be derived from presently known sources using readily available hybridomas or B-cells from non-human host organisms in combination with constant regions derived from, for example, human cell preparations. While the variable region has the advantage of ease of preparation, and the specificity is not affected by its source, the constant region being human is less likely to elicit an immune response from a human subject when the antibodies are administered than would the constant region from a non-human source. However, the definition is not limited to this particular example. In some embodiments, amino acid modifications are made in the variable and/or constant region.

As used herein, “humanized” antibodies refer to forms of non-human (e.g., murine) antibodies that are specific chimeric immunoglobulins, immunoglobulin chains, or fragments thereof (such as Fv, Fab, Fab′, F(ab′)₂, or other antigen-binding subsequences of antibodies) that contain minimal sequence derived from non-human immunoglobulin. For the most part, humanized antibodies are human immunoglobulins (recipient antibody) in which residues from a complementary determining region (CDR) of the recipient are replaced by residues from a CDR of a non-human species (donor antibody), such as mouse, rat, or rabbit, having the desired specificity, affinity, and capacity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one variable domain in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (e.g., an Fc domain), typically that of a human immunoglobulin. Antibodies may have Fc regions modified as described in WO 99/58572. Other forms of humanized antibodies have one or more CDRs (one, two, three, four, five, or six) which are altered with respect to the original antibody, which are also termed one or more CDRs “derived from” one or more CDRs from the original antibody.

As used herein, “human antibody” means an antibody having an amino acid sequence corresponding to that of an antibody produced by a human and/or has been made using any of the techniques for making human antibodies known in the art or disclosed herein. This definition of a human antibody includes antibodies comprising at least one human heavy chain polypeptide or at least one human light chain polypeptide. One such example is an antibody comprising murine light chain and human heavy chain polypeptides. Human antibodies can be produced using various techniques known in the art. In one embodiment, the human antibody is selected from a phage library, where that phage library expresses human antibodies (Vaughan et al., 1996, Nature Biotechnology, 14:309-314; Sheets et al., 1998, PNAS, (USA) 95:6157-6162; Hoogenboom and Winter, 1991, J. Mol. Biol., 227:381; Marks et al., 1991, J. Mol. Biol., 222:581). Human antibodies can also be made by introducing human immunoglobulin loci into transgenic animals, e.g., mice in which the endogenous immunoglobulin genes have been partially or completely inactivated. This approach is described in U.S. Pat. Nos. 5,545,807; 5,545,806; 5,569,825; 5,625,126; 5,633,425; and 5,661,016. Alternatively, the human antibody may be prepared by immortalizing human B-lymphocytes that produce an antibody directed against a target antigen (such B-lymphocytes may be recovered from an individual or may have been immunized in vitro). See, e.g., Cole et al., Monoclonal Antibodies and Cancer Therapy, Alan R. Liss, p. 77 (1985); Boerner et al., 1991, J. Immunol., 147 (1):86-95; and U.S. Pat. No. 5,750,373.

A “variable region” (the term “variable domain” may be used interchangeably herein) of an antibody refers to the variable region of the antibody light chain (VL) or the variable region of the antibody heavy chain (VH), either alone or in combination. The variable regions of the heavy and light chain (VH and VL domains, respectively) each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen-binding site of antibodies. There are at least two techniques for determining CDRs: (1) an approach based on cross-species sequence variability (i.e., Kabat et al. Sequences of Proteins of Immunological Interest, (5th ed., 1991, National Institutes of Health, Bethesda MD)); and (2) an approach based on crystallographic studies of antigen-antibody complexes (Al-lazikani et al (1997) J. Molec. Biol. 273:927-948)). As used herein, a CDR may refer to CDRs defined by either approach or by a combination of both approaches.

A number of HVR delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, MD. (1991)). Chothia refers instead to the location of the structural loops (Chothia and Lesk, J. Mol. Biol. 196:901-917 (1987)). The AbM HVRs represent a compromise between the Kabat HVRs and Chothia structural loops, and are used by Oxford Molecular's AbM antibody modeling software. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B |
| | | | | (Kabat numbering) |
| H1 | H31-H35 | H26-H35 | H26-H32 | H30-H35 |
| | | | | (Chothia numbering) |
| H2 | H50-H65 | H50-H58 | H53-H55 | H47-H58 |
| H3 | H95-H102 | H95-H102 | H96-H101 | H93-H101 |

The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra, or and Edelman, G. M. et al. (1969) Proc. Natl. Acad. Sci. USA 63:78-85).

“Fv” as used herein may refer to the minimum antibody fragment which contains a complete antigen-recognition and -binding site. This fragment typically consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) has the ability to recognize and bind antigen, although at a lower affinity than the entire binding site. “Single-chain Fv” also abbreviated as “sFv” or “scFv” are antibody fragments that comprise the V_{H} and V_{L} antibody domains connected into a single polypeptide chain. Preferably, the sFv polypeptide further comprises a polypeptide linker between the V_{H} and V_{L} domains which enables the sFv to form the desired structure for antigen binding. For a review of the sFv, see Pluckthun in The Pharmacology of Monoclonal Antibodies, vol. 113, Rosenburg and Moore eds., Springer-Verlag, New York, pp. 269-315 (1994).

A “constant region” (the term “constant domain” may be used interchangeably herein) of an antibody refers to the constant region of the antibody light chain (CL) or the constant region of the antibody heavy chain (CH), either alone or in combination. A constant region of an antibody generally provides structural stability and other biological functions such as antibody chain association, secretion, transplacental mobility, and complement binding, but is not involved with binding to the antigen. The amino acid sequence and corresponding exon sequences in the genes of the constant region is dependent upon the species from which it is derived; however, variations in the amino acid sequence leading to allotypes is relatively limited for particular constant regions within a species. In some embodiments, an IgM antibody heavy chain constant region includes a Cμ1, Cμ2, Cμ3, and Cμ4 domain and a tailpiece polypeptide.

The term “Fc region” herein (the term “Fc domain” may be used interchangeably herein) is used to define a C-terminal region of an immunoglobulin heavy chain, including native-sequence Fc regions and variant Fc regions. The boundaries of the Fc region of an immunoglobulin heavy chain might vary. In some embodiments, the Fc region may include the IgM heavy chain constant region domains Cμ2, Cμ3, and Cμ4, and optionally a tailpiece polypeptide. Suitable native-sequence Fc regions for use in the antibodies of the present disclosure include IgM (e.g., human IgM), IgG1, IgG2 (IgG2A, IgG2B), IgG3 and IgG4.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

As used herein, “antibody-dependent cell-mediated cytotoxicity” and “ADCC” refer to a cell-mediated reaction in which nonspecific cytotoxic cells that express Fc receptors (FcRs) (e.g., natural killer (NK) cells, neutrophils, or macrophages) recognize bound antibody on a target cell and subsequently cause lysis of the target cell. ADCC activity of a molecule of interest can be assessed using an in vitro ADCC assay, such as that described in U.S. Pat. No. 5,500,362 or 5,821,337. Useful effector cells for such assays include peripheral blood mononuclear cells (PBMC) and NK cells. Alternatively, or additionally, ADCC activity of the molecule of interest may be assessed in vivo, e.g., in an animal model such as that disclosed in Clynes et al., 1998, PNAS (USA), 95:652-656.

“Complement dependent cytotoxicity” and “CDC” refer to the lysing of a target in the presence of complement. The complement activation pathway is initiated by the binding of the first component of the complement system (C1q) to a molecule (e.g., an antibody) complexed with a cognate antigen. To assess complement activation, a CDC assay, e.g., as described in Gazzano-Santoro et al., J. Immunol. Methods, 202:163 (1996), may be performed.

The terms “polypeptide,” “oligopeptide,” “peptide,” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

“Polynucleotide,” or “nucleic acid,” as used interchangeably herein, refer to polymers of nucleotides of any length, and include DNA and/or RNA. The nucleotides can be deoxyribonucleotides, ribonucleotides, modified nucleotides or bases, and/or their analogs, or any substrate that can be incorporated into a polymer by DNA or RNA polymerase. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after polymerization, such as by conjugation with a labeling component. Other types of modifications include, for example, “caps,” substitution of one or more of the naturally occurring nucleotides with an analog, internucleotide modifications such as, for example, those with uncharged linkages (e.g., methyl phosphonates, phosphotriesters, phosphoamidates, cabamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, α-anomeric sugars, epimeric sugars such as arabinose, xyloses, lyxoses, pyranose sugars, furanose sugars, sedoheptuloses, acyclic analogs, and abasic nucleoside analogs such as methyl ribosides. One or more phosphodiester linkages may be replaced by alternative linking groups. These alternative linking groups include, but are not limited to, embodiments wherein phosphate is replaced by P(O)S(“thioate”), P(S)S (“dithioate”), “(O)NR₂ (“amidate”), P(O)R, P(O)OR′, CO, or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl, or araldyl. Not all linkages in a polynucleotide need be identical. The preceding description applies to all polynucleotides referred to herein, including RNA and DNA.

As used herein, “vector” means a construct that is capable of delivering and desirably expressing one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

As used herein, an “effective dosage,” “effective amount,” or “therapeutically effective amount” of drug, compound, or pharmaceutical composition is an amount sufficient to effect beneficial, desired, and/or therapeutic results. For prophylactic use, beneficial or desired results include results such as eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In the case of treating an individual awaiting a transplantation, for example, an effective amount of the drug may reduce to some extent the level of alloantibodies and/or PRA in the individual. In the case of treating an individual receiving a transplantation or transfusion, an effective amount of the drug may have the effect in and/or relieving to some extent one or more of the symptoms or conditions (such as graft rejection) associated with the transplantation or transfusion. An effective amount can be administered in one or more administrations. For purposes of the present disclosure, an effective amount of drug, compound, or pharmaceutical composition is an amount sufficient to accomplish prophylactic or therapeutic treatment either directly or indirectly. An effective dosage can be administered in one or more administrations. For purposes of the present disclosure, an effective dosage of drug, compound, or pharmaceutical composition is an amount sufficient to accomplish prophylactic or therapeutic treatment either directly or indirectly. As is understood in the clinical context, an effective dosage of a drug, compound, or pharmaceutical composition may or may not be achieved in conjunction with another drug, compound, or pharmaceutical composition. Thus, an effective dosage or amount may be considered in the context of administering one or more therapeutic agents, and a single agent may be considered to be given in an effective amount if, in conjunction with one or more other agents, a desirable result may be or is achieved.

As used herein, “in conjunction with” or “in combination with” refers to administration of one treatment modality in addition to another treatment modality. As such, “in conjunction with” or “in combination with” refer to administration of one treatment modality before, during, or after administration of the other treatment modality to an individual.

As used herein, “treatment” or “treating” is an approach for obtaining beneficial or desired results, including desirably clinical results. Beneficial, desired, and/or therapeutic clinical results include, but are not limited to, one or more of the following: reducing or abrogating one or more symptoms of inflammation or autoimmunity (e.g., stemming from a T-cell mediated inflammatory disease), reducing or abrogating one or more symptoms of a cancer, increasing the likelihood of a successful patient outcome and/or mitigating one or more contraindications or detrimental outcomes related to a medical treatment (e.g., related to a transplantation or transfusion), decreasing symptoms resulting from the disease, increasing the quality of life of those suffering from the disease, decreasing the dose of other medications required to treat the disease, delaying the progression of the disease, and/or prolonging survival of individuals.

As used herein, “delaying development of a disease” means to defer, hinder, slow, retard, stabilize, and/or postpone development of the disease (such as cancer or a T-cell mediated inflammatory disease). This delay can be of varying lengths of time, depending on the history of the disease and/or individual being treated. As is evident to one skilled in the art, a sufficient or significant delay can, in effect, encompass prevention, in that the individual does not develop the disease. For example, a symptom of a cancer, or an inflammatory disease, such as a T-cell mediated inflammatory disease, may be delayed.

An “individual” or a “subject” is a mammal, more desirably a human. Mammals also include, but are not limited to, farm animals, sport animals, pets (such as cats, dogs, or horses), primates, mice, and rats.

As used herein, the terms “binds,” “recognizes,” “specifically recognizes” or “specifically binds” refer to measurable and reproducible interactions such as attraction or binding between a target and an antibody (e.g., a full-length antibody, an antibody fragment, or an antibody VH-VL binding unit) that is determinative of the presence of the target in the presence of a heterogeneous population of molecules including biological molecules. For example, an antibody, antibody fragment, or antibody VH-VL binding unit that binds to an epitope is an antibody that binds this epitope with greater affinity, avidity, more readily, and/or with greater duration than it binds to other epitopes of the target or non-target epitopes. It is also understood by reading this definition that, for example, an antibody, antibody fragment, or antibody VH-VL binding unit that specifically or preferentially binds to a first target may or may not specifically or preferentially bind to a second target. As such, specific binding or preferential binding does not necessarily require (although it can include) exclusive binding. An antibody, antibody fragment, or antibody VH-VL binding unit that binds to a target may have an association constant of greater than or about 10³M⁻¹ or about 10⁴M⁻¹, sometimes about 10⁵M⁻¹ or about 10⁶M⁻¹, in other instances about 10⁶ M⁻¹ or about 10⁷ M⁻¹, about 10⁸M⁻¹ to about 10⁹ M⁻¹, or about 10¹⁰ M⁻¹ to about 10¹¹ M⁻¹ or higher. A variety of immunoassay formats can be used to select antibodies, antibody fragments, or antibody VH-VL binding units that are specifically immunoreactive with a particular protein. For example, solid-phase ELISA immunoassays are routinely used to select monoclonal antibodies specifically immunoreactive with a protein. See, e.g., Harlow and Lane (1988) Antibodies, A Laboratory Manual, Cold Spring Harbor Publications, New York, for a description of immunoassay formats and conditions that can be used to determine specific immunoreactivity.

A “package insert” refers to instructions customarily included in commercial packages of medicaments that contain information about the indications, usage, dosage, administration, contraindications, other medicaments to be combined with the packaged product, and/or warnings concerning the use of such medicaments, etc.

As used herein and in the appended claims, the singular forms “a,” “an,” and “the” include plural reference unless the context clearly indicates otherwise. For example, reference to an “antibody” is a reference to from one to many antibodies, such as molar amounts, and includes equivalents thereof known to those skilled in the art, and so forth.

Reference to “about” a value or parameter herein includes (and describes) embodiments that are directed to that value or parameter per se. For example, description referring to “about X” includes description of “X.”

It is understood that aspects and variations of the present disclosure described herein include “consisting” and/or “consisting essentially of” aspects and variations.

### II. Antibodies of the Disclosure

Certain aspects of the present disclosure relate to antibodies, as well as fragments or variants thereof, that specifically bind to human PSGL-1. In some embodiments, the antibodies of the present disclosure are IgM antibodies. In some embodiments, the antibodies of the present disclosure comprise an IgM constant region, or portions or variants thereof. Exemplary antibodies, antibody fragments, antibody variants, and antibody formats, including IgM antibodies, are described below and in the Examples herein.

Human PSGL-1 may also be referred to as selectin P ligand, SELPG, CLA, CD162, or PSGL1. In some embodiments, an antibody of the present disclosure binds to a polypeptide encoded by the human SELPG gene, e.g., as described by NCBI RefSeq Gene ID No. 6404. In some embodiments, an antibody of the present disclosure binds to a human PSGL-1 polypeptide, or a fragment thereof. In some embodiments, an antibody of the present disclosure binds to a polypeptide comprising the amino acid sequence of SEQ ID NO: 21, or a fragment thereof. In some embodiments, an antibody of the present disclosure binds to a polypeptide comprising the amino acid sequence of SEQ ID NO: 22, or a fragment thereof. In some embodiments, an antibody of the present disclosure binds to a polypeptide comprising the amino acid sequence of SEQ ID NO: 21 and binds to a polypeptide comprising the amino acid sequence of SEQ ID NO:22, or fragments thereof. The amino acid sequence of SEQ ID NO:21 depicts full length human PSGL-1, GenBank™ accession number AAA74577.1, and the amino acid sequence of SEQ ID NO:22 depicts the shorter 402 amino acid human PSGL-1 protein (GenBank™ accession number XP_005269133). In specific embodiments, an antibody described herein specifically binds to human PSGL-1 as determined, e.g., by ELISA or other antigen-binding assays known in the art, or described herein.

In some embodiments, the antibodies of the present disclosure are IgM antibodies. As is known in the art, IgM antibodies are the first immunoglobulins produced by B cells in response to stimulation by antigen, and are present at around 1.5 mg/ml in serum with a half-life of about 5 days. IgM antibodies form polymers where multiple immunoglobulins are covalently linked together with disulfide bonds. IgM mostly exists as a pentamer but also as a hexamer and therefore typically contains 10 or 12 antigen binding sites. The pentameric form typically contains an additional polypeptide, called the J-chain, but can also be made in the absence of J-chain. Due to its polymeric nature, IgM typically possesses high avidity. An IgM binding unit typically includes two light chains and two heavy chains. While IgG contains three heavy chain constant domains (CH1, CH2 and CH3), the heavy chain of IgM antibodies additionally contains a fourth constant domain that can include a C-terminal “tailpiece” polypeptide. The constant domains of the heavy chain of IgM antibodies are termed Cμ1, Cμ2, Cμ3, and Cμ4. An exemplary human IgM heavy chain constant region comprises the amino acid sequence of SEQ ID NO: 18. In some cases, an IgM binding unit comprises an IgM antibody heavy chain constant region, comprising at least a Cμ4 constant domain, fused to a variable domain sequence (VH), with or without an associated antibody light chain variable domain (VL) sequence. Anti-PSGL-1 antibodies of the disclosure may be mono-, or multi-specific (including bispecific) IgM molecules. Exemplary IgM antibodies are further described in PCT application PCT/US2014/054079.

As discussed above, five IgM binding units can form a complex with an additional small polypeptide chain called the J-chain to form a pentameric IgM antibody. An exemplary J-chain polypeptide comprises the amino acid sequence of SEQ ID NO: 19. Without the J-chain, IgM binding units typically assemble into a hexamer. Without wishing to be bound by theory, the assembly of IgM binding units into a hexameric or pentameric antibodies is thought to involve the Cμ3, Cμ4 and tailpiece domains. Accordingly, a hexameric or pentameric IgM antibody provided herein may include IgM constant regions that include at least the Cμ3, Cμ4, and tailpiece domains, and may further include the Cμ1 and Cμ2 domains, as well as fragments or variants thereof. In some embodiments, an IgM antibody of the present disclosure may include an IgM heavy chain constant region (e.g., from humans), for example, as set forth in SEQ ID NO: 18, or a variant, derivative, or analog thereof. In some embodiments, an IgM antibody of the present disclosure may include a complete IgM heavy chain constant region (e.g., from humans), or a variant, derivative, or analog thereof.

In some embodiments, provided herein is an anti-PSGL-1 antibody, e.g., an IgM antibody, that comprises at least one, at least two, or all three light chain variable domain CDR sequences selected from (a) a CDR-L1 comprising the amino acid sequence of SEQ ID NO: 1; (b) a CDR-L2 comprising the amino acid sequence of SEQ ID NO: 2; and (c) a CDR-L3 comprising the amino acid sequence of SEQ ID NO: 3. In some embodiments, the anti-PSGL-1 antibody comprises (a) a CDR-L1 comprising the amino acid sequence of SEQ ID NO: 1; (b) a CDR-L2 comprising the amino acid sequence of SEQ ID NO: 2; and (c) a CDR-L3 comprising the amino acid sequence of SEQ ID NO: 3, e.g., as shown in Table 1.

In some embodiments, provided herein is an anti-PSGL-1 antibody, e.g., and IgM antibody, that comprises at least one, at least two, or all three heavy chain variable domain CDR sequences selected from (a) a CDR-H1 comprising the amino acid sequence of SEQ ID NO: 4; (b) a CDR-H2 comprising the amino acid sequence of SEQ ID NO: 5; and (c) a CDR-H3 comprising the amino acid sequence of SEQ ID NO: 6. In some embodiments, the anti-PSGL-1 antibody comprises (a) a CDR-H1 comprising the amino acid sequence of SEQ ID NO: 4; (b) a CDR-H2 comprising the amino acid sequence of SEQ ID NO: 5; and (c) a CDR-H3 comprising the amino acid sequence of SEQ ID NO: 6, e.g., as shown in Table 1.

In some embodiments, provided herein is an anti-PSGL-1 antibody, e.g., an IgM antibody, that comprises (a) a light chain variable domain comprising at least one, at least two, or all three light chain variable domain CDR sequences selected from (i) a CDR-L1 comprising the amino acid sequence of SEQ ID NO: 1; (ii) a CDR-L2 comprising the amino acid sequence of SEQ ID NO: 2; and (iii) a CDR-L3 comprising the amino acid sequence of SEQ ID NO: 3; and (b) a heavy chain variable domain comprising at least one, at least two, or all three heavy chain variable domain CDR sequences selected from (i) a CDR-H1 comprising the amino acid sequence of SEQ ID NO: 4; (ii) a CDR-H2 comprising the amino acid sequence of SEQ ID NO: 5; and (iii) a CDR-H3 comprising the amino acid sequence of SEQ ID NO: 6, e.g., as shown in Table 1.

In some embodiments, the anti-PSGL-1 antibody comprises at least one of (a) a CDR-H3 comprising the amino acid sequence of SEQ ID NO: 6; and (b) a CDR-L1 comprising the amino acid sequence of SEQ ID NO: 1, e.g., as shown in Table 1. In some embodiments, the anti-PSGL-1 antibody comprises a CDR-H3 comprising the amino acid sequence of SEQ ID NO: 6. In some embodiments, the anti-PSGL-1 antibody comprises a CDR-L1 comprising the amino acid sequence of SEQ ID NO: 1. In some embodiments, the anti-PSGL-1 antibody comprises (a) a CDR-H3 comprising the amino acid sequence of SEQ ID NO: 6; and (b) a CDR-L1 comprising the amino acid sequence of SEQ ID NO: 1.

In some embodiments, provided herein is an anti-PSGL-1 antibody, e.g., an IgM antibody, that comprises (a) a heavy chain variable domain that comprises a CDR-H1 comprising the amino acid sequence of SEQ ID NO: 4, a CDR-H2 comprising the amino acid sequence of SEQ ID NO: 5, and a CDR-H3 comprising the amino acid sequence of SEQ ID NO: 6; and (b) a light chain variable domain that comprises a CDR-L1 comprising the amino acid sequence of SEQ ID NO: 1, a CDR-L2 comprising the amino acid sequence of SEQ ID NO: 2, and a CDR-L3 comprising the amino acid sequence of SEQ ID NO: 3, e.g., as shown in Table 1.

Exemplary anti-PSGL-1 antibody sequences, as well as exemplary human PSGL-1 polypeptide sequences, are provided in Table 1.

TABLE 1
Exemplary anti-PSGL-1 antibody and human PSGL-1 polypeptide sequences.

| Description | Sequence | SEQ ID NO |
|---|---|---|
| 15A7H-IgM CDR-L1 | RSSQSIVHNDGNTYFE | 1 |
| amino acid sequence | | |
| 15A7H-IgM CDR-L2 | KVSNRFS | 2 |
| amino acid sequence | | |
| 15A7H-IgM CDR-L3 | FQGSYVPLT | 3 |
| amino acid sequence | | |
| 15A7H-IgM CDR-H1 | SFGMH | 4 |
| amino acid sequence | | |
| 15A7H-IgM CDR-H2 | YINGGSSTIFYANAVKG | 5 |
| amino acid sequence | | |
| 15A7H-IgM CDR-H3 | YASYGGGAMDY | 6 |
| amino acid sequence | | |
| 15A 7H-IgM VL FR1 | DIQMTQSPSSLSASVGDRVTITC | 7 |
| amino acid sequence | | |
| 15A 7H-IgM VL FR2 | WYQQKPGKAPKLLIY | 8 |
| amino acid sequence | | |
| 15A 7H-IgM VL FR3 | GVPSRFSGSGSGTHFTLTISSLQPEDFATYYC | 9 |
| amino acid sequence | | |
| 15A 7H-IgM VL FR4 | FGQGTKVEIK | 10 |
| amino acid sequence | | |
| 15A7H-IgM VH FR1 | EVQLVESGGGLVQPGGSLRLSCAASGFTFS | 11 |
| amino acid sequence | | |
| 15A7H-IgM VH FR2 | WVRQAPGKGLEWVA | 12 |
| amino acid sequence | | |
| 15A7H-IgM VH FR3 | RFTISRDNAKNTLYLQMNSLRAEDTAVYYCAR | 13 |
| amino acid sequence | | |
| 15A7H-IgM VH FR4 | WGQGTLVTVSS | 14 |
| amino acid sequence | | |
| 15A7H-IgM VL | DIQMTQSPSSLSASVGDRVTITCRSSQSIVHNDGNTY | 15 |
| domain amino acid | FEWYQQKPGKAPKLLIYKVSNRFSGVPSRFSGSGSG | |
| sequence | THFTLTISSLQPEDFATYYCFQGSYVPLTFGQGTKVEI | |
| | K | |
| 15A7H-IgM VH | EVQLVESGGGLVQPGGSLRLSCAASGFTFSSFGMHW | 16 |
| domain amino acid | VRQAPGKGLEWVAYINGGSSTIFYANAVKGRFTISR | |
| sequence | DNAKNTLYLQMNSLRAEDTAVYYCARYASYGGGA | |
| | MDYWGQGTLVTVSS | |
| 15A 7H-IgM Light | DIQMTQSPSSLSASVGDRVTITCRSSQSIVHNDGNTY | 17 |
| chain amino acid | FEWYQQKPGKAPKLLIYKVSNRFSGVPSRFSGSGSG | |
| Sequence | THFTLTISSLQPEDFATYYCFQGSYVPLTFGQGTKVEI | |
| | KRTVAAPSVFIFPPSDEQLKSGTASVVCLLNNFYPRE | |
| | AKVQWKVDNALQSGNSQESVTEQDSKDSTYSLSST | |
| | LTLSKADYEKHKVYACEVTHQGLSSPVTKSFNRGEC | |
| 15A7H-IgM heavy | GSASAPTLFPLVSCENSPSDTSSVAVGCLAQDFLPDSI | 18 |
| chain constant region | TFSWKYKNNSDISSTRGFPSVLRGGKYAATSQVLLPS | |
| | KDVMQGTDEHVVCKVQHPNGNKEKNVPLPVIAELP | |
| | PKVSVFVPPRDGFFGNPRKSKLICQATGFSPRQIQVS | |
| | WLREGKQVGSGVTTDQVQAEAKESGPTTYKVTSTL | |
| | TIKESDWLSQSMFTCRVDHRGLTFQQNASSMCVPDQ | |
| | DTAIRVFAIPPSFASIFLTKSTKLTCLVTDLTTYDSVTI | |
| | SWTRQNGEAVKTHTNISESHPNATFSAVGEASICED | |
| | DWNSGERFTCTVTHTDLPSPLKQTISRPKGVALHRP | |
| | DVYLLPPAREQLNLRESATITCLVTGFSPADVFVQW | |
| | MQRGQPLSPEKYVTSAPMPEPQAPGRYFAHSILTVSE | |
| | EEWNTGETYTCVVAHEALPNRVTERTVDKSTGKPT | |
| | LYNVSLVMSDTAGTCY | |
| 15A7H-IgM J-chain | QEDERIVLVDNKCKCARITSRIIRSSEDPNEDIVERNIR | 19 |
| | IIVPLNNRENISDPTSPLRTRFVYHLSDLCKKCDPTEV | |
| | ELDNQIVTATQSNICDEDSATETCYTYDRNKCYTAV | |
| | VPLVYGGETKMVETALTPDACYPD | |
| 15A7H-IgM heavy | EVQLVESGGGLVQPGGSLRLSCAASGFTFSSFGMHW | 20 |
| chain amino acid | VRQAPGKGLEWVAYINGGSSTIFYANAVKGRFTISR | |
| sequence | DNAKNTLYLQMNSLRAEDTAVYYCARYASYGGGA | |
| | MDYWGQGTLVTVSSGSASAPTLFPLVSCENSPSDTS | |
| | SVAVGCLAQDFLPDSITFSWKYKNNSDISSTRGFPSV | |
| | LRGGKYAATSQVLLPSKDVMQGTDEHVVCKVQHP | |
| | NGNKEKNVPLPVIAELPPKVSVFVPPRDGFFGNPRKS | |
| | KLICQATGFSPRQIQVSWLREGKQVGSGVTTDQVQA | |
| | EAKESGPTTYKVTSTLTIKESDWLSQSMFTCRVDHR | |
| | GLTFQQNASSMCVPDQDTAIRVFAIPPSFASIFLTKST | |
| | KLTCLVTDLTTYDSVTISWTRQNGEAVKTHTNISESH | |
| | PNATFSAVGEASICEDDWNSGERFTCTVTHTDLPSPL | |
| | KQTISRPKGVALHRPDVYLLPPAREQLNLRESATITC | |
| | LVTGFSPADVFVQWMQRGQPLSPEKYVTSAPMPEP | |
| | QAPGRYFAHSILTVSEEEWNTGETYTCVVAHEALPN | |
| | RVTERTVDKSTGKPTLYNVSLVMSDTAGTCY | |
| Amino acid sequence | MPLQLLLLLILLGPGNSLQLWDTWADEAEKALGPLL | 21 |
| of human PSGL-1 | ARDRRQATEYEYLDYDFLPETEPPEMLRNSTDTTPL | |
| | TGPGTPESTTVEPAARRSTGLDAGGAVTELTTELAN | |
| | MGNLSTDSAAMEIQTTQPAATEAQTTQPVPTEAQTT | |
| | PLAATEAQTTRLTATEAQTTPLAATEAQTTPPAATE | |
| | AQTTQPTGLEAQTTAPAAMEAQTTAPAAMEAQTTP | |
| | PAAMEAQTTQTTAMEAQTTAPEATEAQTTQPTATE | |
| | AQTTPLAAMEALSTEPSATEALSMEPTTKRGLFIPFS | |
| | VSSVTHKGIPMAASNLSVNYPVGAPDHISVKQCLLAI | |
| | LILALVATIFFVCTVVLAVRLSRKGHMYPVRNYSPTE | |
| | MVCISSLLPDGGEGPSATANGGLSKAKSPGLTPEPRE | |
| | DREGDDLTLHSFLP | |
| PSGL-1 variant amino | MPLQLLLLLILLGPGNSLQLWDTWADEAEKALGPLL | 22 |
| acid sequence | ARDRRQATEYEYLDYDFLPETEPPEMLRNSTDTTPL | |
| | TGPGTPESTTVEPAARRSTGLDAGGAVTELTTELAN | |
| | MGNLSTDSAAMEIQTTQPAATEAQTTPLAATEAQTT | |
| | RLTATEAQTTPLAATEAQTTPPAATEAQTTQPTGLE | |
| | AQTTAPAAMEAQTTAPAAMEAQTTPPAAMEAQTTQ | |
| | TTAMEAQTTAPEATEAQTTQPTATEAQTTPLAAMEA | |
| | LSTEPSATEALSMEPTTKRGLFIPFSVSSVTHKGIPMA | |
| | ASNLSVNYPVGAPDHISVKQCLLAILILALVATIFFVC | |
| | TVVLAVRLSRKGHMYPVRNYSPTEMVCISSLLPDGG | |
| | EGPSATANGGLSKAKSPGLTPEPREDREGDDLTLHSF | |
| | LP | |
| 15A7H-IgG4 Light | DIQMTQSPSSLSASVGDRVTITCRSSQSIVHNDGNTY | 23 |
| Chain amino acid | FEWYQQKPGKAPKLLIYKVSNRFSGVPSRFSGSGSG | |
| sequence | THFTLTISSLQPEDFATYYCFQGSYVPLTFGQGTKVEI | |
| | KRTVAAPSVFIFPPSDEQLKSGTASVVCLLNNFYPRE | |
| | AKVQWKVDNALQSGNSQESVTEQDSKDSTYSLSST | |
| | LTLSKADYEKHKVYACEVTHQGLSSPVTKSFNRGEC | |
| 15A7H-IgG4 Heavy | EVQLVESGGGLVQPGGSLRLSCAASGFTFSSFGMHW | 24 |
| Chain amino acid | VRQAPGKGLEWVAYINGGSSTIFYANAVKGRFTISR | |
| sequence | DNAKNTLYLQMNSLRAEDTAVYYCARYASYGGGA | |
| | MDYWGQGTLVTVSSASTKGPSVFPLAPCSRSTSEST | |
| | AALGCLVKDYFPEPVTVSWNSGALTSGVHTFPAVLQ | |
| | SSGLYSLSSVVTVPSSSLGTKTYTCNVDHKPSNTKVD | |
| | KRVESKYGPPCPPCPAPEFLGGPSVFLFPPKPKDTLMI | |
| | SRTPEVTCVVVDVSQEDPEVQFNWYVDGVEVHNAK | |
| | TKPREEQFNSTYRVVSVLTVLHQDWLNGKEYKCKV | |
| | SNKGLPSSIEKTISKAKGQPREPQVYTLPPSQEEMTK | |
| | NQVSLTCLVKGFYPSDIAVEWESNGQPENNYKTTPP | |
| | VLDSDGSFFLYSRLTVDKSRWQEGNVFSCSVMHEAL | |
| | HNHYTQKSLSLSLGK | |

In some embodiments, the anti-PSGL-1 antibody, e.g., the IgM antibody, is a humanized anti-PSGL1 antibody. In some embodiments, the anti-PSGL-1 antibody comprises CDRs as in any of the embodiments provided herein, and further comprises a human immunoglobulin framework or a human consensus framework. In some embodiments, the humanized anti-PSGL1 antibody comprises (a) a heavy chain variable domain that comprises a CDR-H1 comprising the amino acid sequence of SEQ ID NO: 4, a CDR-H2 comprising the amino acid sequence of SEQ ID NO: 5, and a CDR-H3 comprising the amino acid sequence of SEQ ID NO: 6; and (b) a light chain variable domain (VL) that comprises a CDR-L1 comprising the amino acid sequence of SEQ ID NO: 1, a CDR-L2 comprising the amino acid sequence of SEQ ID NO: 2, and a CDR-L3 comprising the amino acid sequence of SEQ ID NO: 3, e.g., as shown in Table 1.

In some embodiments, the anti-PSGL-1 antibody comprises at least one, two, three, four, five, six, seven, or eight framework regions (FRs) selected from (a) a light chain variable domain (VL) FRI comprising the amino acid sequence of SEQ ID NO: 7; (b) a VL FR2 comprising the amino acid sequence of SEQ ID NO: 8; (c) a VL FR3 comprising the amino acid sequence of SEQ ID NO: 9; (d) a VL FR4 comprising the amino acid sequence of SEQ ID NO: 10, (e) a heavy chain variable domain (VH) FRI comprising the amino acid sequence of SEQ ID NO: 11; (f) a VH FR2 comprising the amino acid sequence of SEQ ID NO: 12; (g) a VH FR3 comprising the amino acid sequence of SEQ ID NO: 13; and (h) a VH FR4 comprising the amino acid sequence of SEQ ID NO: 14, e.g., as shown in Table 1.

In some embodiments, the anti-PSGL-1 antibody comprises at least one, two, three, or all four light chain variable domain (VL) FR sequences selected from (a) a VL FR1 comprising the amino acid sequence of SEQ ID NO: 7; (b) a VL FR2 comprising the amino acid sequence of SEQ ID NO: 8; (c) a VL FR3 comprising the amino acid sequence of SEQ ID NO: 9; and (d) a VL FR4 comprising the amino acid sequence of SEQ ID NO: 10. In some embodiments, the anti-PSGL-1 antibody comprises at least one, two, three, or all four heavy chain variable domain (VH) FR sequences selected from (a) a VH FRI comprising the amino acid sequence of SEQ ID NO: 11; (b) a VH FR2 comprising the amino acid sequence of SEQ ID NO: 12; (c) a VH FR3 comprising the amino acid sequence of SEQ ID NO: 13; and (d) a VH FR4 comprising the amino acid sequence of SEQ ID NO: 14.

In some embodiments, the anti-PSGL-1 antibody comprises (a) a VL FRI comprising the amino acid sequence of SEQ ID NO: 7; (b) a VL FR2 comprising the amino acid sequence of SEQ ID NO: 8; (c) a VL FR3 comprising the amino acid sequence of SEQ ID NO: 9; and (d) a VL FR4 comprising the amino acid sequence of SEQ ID NO: 10. In some embodiments, the anti-PSGL-1 antibody comprises (a) a VH FRI comprising the amino acid sequence of SEQ ID NO: 11; (b) a VH FR2 comprising the amino acid sequence of SEQ ID NO: 12; (c) a VH FR3 comprising the amino acid sequence of SEQ ID NO: 13; and (d) a VH FR4 comprising the amino acid sequence of SEQ ID NO: 14.

In some embodiments, the anti-PSGL-1 antibody comprises a (a) VL domain comprising at least one, at least two, at least three, or all four VL FR sequences selected from (i) a VL FR1 comprising the amino acid sequence of SEQ ID NO: 7; (ii) a VL FR2 comprising the amino acid sequence of SEQ ID NO: 8; (iii) a VL FR3 comprising the amino acid sequence of SEQ ID NO: 9; and (iv) a VL FR4 comprising the amino acid sequence of SEQ ID NO: 10; and (b) a VH domain comprising at least one, at least two, at least three, or all four VH FR sequences selected from (i) a VH FRI comprising the amino acid sequence of SEQ ID NO: 11; (ii) a VH FR2 comprising the amino acid sequence of SEQ ID NO: 12; (iii) a VH FR3 comprising the amino acid sequence of SEQ ID NO: 13; and (iv) a VH FR4 comprising the amino acid sequence of SEQ ID NO: 14.

In some embodiments, the anti-PSGL-1 antibody comprises (a) a VL domain comprising (i) a VL FRI comprising the amino acid sequence of SEQ ID NO: 7; (ii) a VL FR2 comprising the amino acid sequence of SEQ ID NO: 8; (iii) a VL FR3 comprising the amino acid sequence of SEQ ID NO: 9; and (iv) a VL FR4 comprising the amino acid sequence of SEQ ID NO: 10; and (b) a VH domain comprising (i) a VH FRI comprising the amino acid sequence of SEQ ID NO: 11; (ii) a VH FR2 comprising the amino acid sequence of SEQ ID NO: 12; (iii) a VH FR3 comprising the amino acid sequence of SEQ ID NO: 13; and (iv) a VH FR4 comprising the amino acid sequence of SEQ ID NO: 14.

In some embodiments, the anti-PSGL-1 antibody comprises (a) a VL domain comprising (i) a VL FRI comprising the amino acid sequence of SEQ ID NO: 7; (ii) a CDR-L1 comprising the amino acid sequence of SEQ ID NO: 1; (iii) a VL FR2 comprising the amino acid sequence of SEQ ID NO: 8; (iv) a CDR-L2 comprising the amino acid sequence of SEQ ID NO: 2; (v) a VL FR3 comprising the amino acid sequence of SEQ ID NO: 9; (vi) a CDR-L3 comprising the amino acid sequence of SEQ ID NO: 3; and (vii) a VL FR4 comprising the amino acid sequence of SEQ ID NO: 10. In some embodiments, the anti-PSGL-1 antibody comprises (a) a VH domain comprising (i) a VH FRI comprising the amino acid sequence of SEQ ID NO: 11; (ii) a CDR-H1 comprising the amino acid sequence of SEQ ID NO: 4; (iii) a VH FR2 comprising the amino acid sequence of SEQ ID NO: 12; (iv) a CDR-H2 comprising the amino acid sequence of SEQ ID NO: 5; (v) a VH FR3 comprising the amino acid sequence of SEQ ID NO: 13; (vi) a CDR-H3 comprising the amino acid sequence of SEQ ID NO: 6; and (vii) a VH FR4 comprising the amino acid sequence of SEQ ID NO: 14.

In some embodiments, the anti-PSGL-1 antibody comprises (a) a VL domain comprising (i) a VL FRI comprising the amino acid sequence of SEQ ID NO: 7; (ii) a CDR-L1 comprising the amino acid sequence of SEQ ID NO: 1; (iii) a VL FR2 comprising the amino acid sequence of SEQ ID NO: 8; (iv) a CDR-L2 comprising the amino acid sequence of SEQ ID NO: 2; (v) a VL FR3 comprising the amino acid sequence of SEQ ID NO: 9; (vi) a CDR-L3 comprising the amino acid sequence of SEQ ID NO: 3; and (vii) a VL FR4 comprising the amino acid sequence of SEQ ID NO: 10; and (b) a VH domain comprising (viii) a VH FRI comprising the amino acid sequence of SEQ ID NO: 11; (ix) a CDR-H1 comprising the amino acid sequence of SEQ ID NO: 4; (x) a VH FR2 comprising the amino acid sequence of SEQ ID NO: 12; (xi) a CDR-H2 comprising the amino acid sequence of SEQ ID NO: 5; (xii) a VH FR3 comprising the amino acid sequence of SEQ ID NO: 13; (xiii) a CDR-H3 comprising the amino acid sequence of SEQ ID NO: 6; and (xiv) a VH FR4 comprising the amino acid sequence of SEQ ID NO: 14.

In some embodiments, the anti-PSGL-1 antibody comprises a heavy chain variable domain (VH) comprising a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 16. In some embodiments, a VH sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the amino acid sequence of SEQ ID NO: 16 contains substitutions (e.g., conservative substitutions), insertions, or deletions relative to the reference sequence, but an anti-PSGL-1 antibody comprising that sequence retains the ability to bind to PSGL-1. In some embodiments, a total of 1 to 10 amino acids have been substituted, inserted and/or deleted in SEQ ID NO: 16. In some embodiments, a total of 1 to 5 amino acids have been substituted, inserted and/or deleted in SEQ ID NO: 16. In some embodiments, substitutions, insertions, or deletions occur in regions outside the CDRs (i.e., in the FRs). In some embodiments, the anti-PSGL-1 antibody comprises the VH sequence of SEQ ID NO: 16, including post-translational modifications of that sequence. In some embodiments, the VH comprises one, two or three CDRs selected from: (a) a CDR-H1 comprising the amino acid sequence of SEQ ID NO: 4; (b) a CDR-H2 comprising the amino acid sequence of SEQ ID NO: 5; and (c) a CDR-H3 comprising the amino acid sequence of SEQ ID NO: 6.

(SEQ ID NO: 16)
EVQLVESGGGLVQPGGSLRLSCAASGFTFSSFGMHWVRQAPGKGL
EWVAYINGGSSTIFYANAVKGRFTISRDNAKNTLYLQMNSLRAED
TAVYYCARYASYGGGAMDYWGQGTLVTVSS

In some embodiments, the anti-PSGL-1 antibody comprises a light chain variable domain (VL) having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 15. In certain embodiments, a VL sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the amino acid sequence of SEQ ID NO: 15 contains substitutions (e.g., conservative substitutions), insertions, or deletions relative to the reference sequence, but an anti-PSGL-1 antibody comprising that sequence retains the ability to bind to PSGL-1. In certain embodiments, a total of 1 to 10 amino acids have been substituted, inserted and/or deleted in SEQ ID NO: 15. In certain embodiments, a total of 1 to 5 amino acids have been substituted, inserted and/or deleted in SEQ ID NO: 15. In certain embodiments, the substitutions, insertions, or deletions occur in regions outside the CDRs (i.e., in the FRs). In some embodiments, the anti-PSGL-1 antibody comprises the VL sequence of SEQ ID NO: 15, including post-translational modifications of that sequence. In some embodiments, the VL comprises one, two or three CDRs selected from: (a) a CDR-L1 comprising the amino acid sequence of SEQ ID NO: 1; (b) a CDR-L2 comprising the amino acid sequence of SEQ ID NO: 2; and (c) a CDR-L3 comprising the amino acid sequence of SEQ ID NO: 3.

(SEQ ID NO: 15)
DIQMTQSPSSLSASVGDRVTITCRSSQSIVHNDGNTYFEWYQQKP
GKAPKLLIYKVSNRFSGVPSRFSGSGSGTHFTLTISSLQPEDFAT
YYCFQGSYVPLTFGQGTKVEIK

In some embodiments, the anti-PSGL-1 antibody comprises a VH as in any of the embodiments provided herein, and a VL as in any of the embodiments provided herein. In some embodiments, the anti-PSGL-1 antibody comprises the VH and the VL sequences in SEQ ID NO: 16 and SEQ ID NO: 15, respectively, including post-translational modifications of those sequences.

In some embodiments, the anti-PSGL-1 antibody comprises an anti-PSGL-1 antibody that binds to the same epitope as an anti-PSGL-1 antibody provided herein. For example, in some embodiments, the anti-PSGL-1 antibody binds to the same epitope as an anti-PSGL-1 antibody comprising a VH comprising the sequence in SEQ ID NO: 16 and a VL comprising the sequence of SEQ ID NO: 15.

In some embodiments, the anti-PSGL-1 antibody is a monoclonal antibody, a chimeric antibody, humanized antibody, or human antibody. In some embodiments, the anti-PSGL-1 antibody is an antigen-binding fragment of an anti-PSGL-1 antibody described herein, e.g., a Fv, Fab, Fab′, scFv, diabody, or F(ab′)2 fragment.

In some embodiments, the anti-PSGL-1 antibody comprises a substantially full length anti-PSGL-1 antibody, e.g., an IgM antibody or other antibody class or isotype. In some embodiments, the anti-PSGL-1 antibody is of the IgG, IgM, or IgA class. In some embodiments, the anti-PSGL-1 antibody has a human IgG1, IgG2, IgG3, or IgG4 constant domain. In some embodiments, the anti-PSGL-1 antibody is of the IgM class. In some embodiments, the anti-PSGL-1 antibody comprises an IgM constant domain.

In some embodiments, the anti-PSGL-1 antibody of the present disclosure comprises a light chain comprising a kappa CL domain. In some embodiments, the light chain comprises a human kappa light chain.

In some embodiments, the anti-PSGL-1 antibody comprises a heavy chain comprising the amino acid sequence of SEQ ID NO: 20, or a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity thereto, and/or a light chain comprising the amino acid sequence of SEQ ID NO: 17, or a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity thereto. In some embodiments, the anti-PSGL-1 antibody comprises a heavy chain comprising the amino acid sequence of SEQ ID NO: 20, and a light chain comprising the amino acid sequence of SEQ ID NO: 17.

(SEQ ID NO: 17)
DIQMTQSPSSLSASVGDRVTITCRSSQSIVHNDGNTYFEWYQQKP
GKAPKLLIYKVSNRFSGVPSRFSGSGSGTHFTLTISSLQPEDFAT
YYCFQGSYVPLTFGQGTKVEIKRTVAAPSVFIFPPSDEQLKSGTA
SVVCLLNNFYPREAKVQWKVDNALQSGNSQESVTEQDSKDSTYSL
SSTLTLSKADYEKHKVYACEVTHQGLSSPVTKSFNRGEC
(SEQ ID NO: 20)
EVQLVESGGGLVQPGGSLRLSCAASGFTFSSFGMHWVRQAPGKGL
EWVAYINGGSSTIFYANAVKGRFTISRDNAKNTLYLQMNSLRAED
TAVYYCARYASYGGGAMDYWGQGTLVTVSSGSASAPTLFPLVSCE
NSPSDTSSVAVGCLAQDFLPDSITFSWKYKNNSDISSTRGFPSVL
RGGKYAATSQVLLPSKDVMQGTDEHVVCKVQHPNGNKEKNVPLPV
IAELPPKVSVFVPPRDGFFGNPRKSKLICQATGFSPRQIQVSWLR
EGKQVGSGVTTDQVQAEAKESGPTTYKVTSTLTIKESDWLSQSMF
TCRVDHRGLTFQQNASSMCVPDQDTAIRVFAIPPSFASIFLTKST
KLTCLVTDLTTYDSVTISWTRQNGEAVKTHTNISESHPNATFSAV
GEASICEDDWNSGERFTCTVTHTDLPSPLKQTISRPKGVALHRPD
VYLLPPAREQLNLRESATITCLVTGFSPADVFVQWMQRGQPLSPE
KYVTSAPMPEPQAPGRYFAHSILTVSEEEWNTGETYTCVVAHEAL
PNRVTERTVDKSTGKPTLYNVSLVMSDTAGTCY

In some embodiments, the anti-PSGL-1 antibody of the present disclosure comprises, according to numbering in Kabat et al., the CDR-H1, CDR-H2, CDR-H3, CDR-L1, CDR-L2, and/or CDR-L3 of the anti-PSGL-1 antibody 15A7H. In some embodiments, the anti-PSGL-1 antibody of the present disclosure comprises the CDR-H1, CDR-H2, CDR-H3, CDR-L1, CDR-L2, and CDR-L3 of the anti-PSGL-1 antibody 15A7H. In some embodiments, the anti-PSGL-1 antibody of the present disclosure comprises the VH domain and/or the VL domain of the anti-PSGL-1 antibody 15A7H. In some embodiments, the anti-PSGL-1 antibody of the present disclosure comprises the VH domain and the VL domain of the anti-PSGL-1 antibody 15A7H. In some embodiments, the anti-PSGL-1 antibody of the present disclosure comprises the heavy chain and/or the light chain of the anti-PSGL-1 antibody 15A7H, or portions or variants thereof. For example, in some embodiments, the anti-PSGL-1 antibody of the present disclosure comprises the heavy chain of the anti-PSGL-1 antibody 15A7H formatted on an IgM heavy chain, e.g., a human IgM heavy chain. In some embodiments, the anti-PSGL-1 antibody of the present disclosure comprises the heavy chain and the light chain of the anti-PSGL-1 antibody 15A7H, or portions or variants thereof. In some embodiments, the anti-PSGL-1 antibody of the present disclosure comprises the light chain of the anti-PSGL-1 antibody 15A7H, and the heavy chain of the anti-PSGL-1 antibody 15A7H formatted on an IgM heavy chain, e.g., a human IgM heavy chain. The anti-PSGL-1 antibody 15A7H is described in International App. Pub. No. WO2012/174001.

An anti-PSGL-1 IgM antibody of the disclosure may be pentameric or hexameric, comprising five or six IgM binding units, respectively, one or more of which can specifically bind to PSGL-1. An IgM binding unit typically includes two light chains and two heavy chains. However, a binding unit need not include full-length antibody heavy and light chains, but will typically be bivalent, i.e., will include two antigen binding domains. Certain IgM antibodies of the disclosure are pentameric or hexameric and include five or six bivalent binding units, respectively, that optionally include IgM constant regions, or fragments thereof. In some embodiments, the IgM antibodies provided herein comprise a pentameric or hexameric IgM antibody comprising five or six bivalent binding units, respectively, where each binding unit includes two IgM heavy chain constant regions or fragments or variants thereof as provided herein.

In some embodiments, an anti-PSGL-1 IgM antibody of the disclosure may comprise an IgM heavy chain constant region comprising one or more of a Cμ1, Cμ2, Cμ3, and/or Cμ4 domain. In some embodiments, the heavy chain constant region can serve a desired function in the IgM antibody, e.g., associate with a second IgM constant region to form a binding unit, or associate with other binding units to form a hexamer or a pentamer. In some embodiments, an IgM antibody of the disclosure further comprises a tailpiece polypeptide.

In some embodiments, the two IgM heavy chain constant regions, or fragments or variants thereof, within an individual binding unit of an IgM antibody of the disclosure each comprise a Cμ3 domain or a fragment or variant thereof, a Cμ4 domain or a fragment or variant thereof, a tailpiece (TP) or fragment or variant thereof, or any combination of a Cμ3 domain, a Cμ4 domain, and a TP, or fragments or variants thereof. In some embodiments, the two IgM heavy chain constant regions, or fragments or variants thereof, within an individual binding unit of an IgM antibody of the disclosure further comprise a Cμ1 domain or a fragment or variant thereof, and/or a Cμ2 domain or a fragment or variant thereof. In some embodiments, the two IgM heavy chain constant regions in a given binding unit of an IgM antibody of the disclosure are associated with an antigen binding domain, for example a heavy chain variable region (VH) and a light chain variable region (VL) or an Fv portion of an antibody, e.g., as described herein. In some embodiments, an IgM antibody of the present disclosure comprises an IgM heavy chain constant region comprising the amino acid sequence of SEQ ID NO: 18, or a fragment thereof, or an amino acid sequence with at least about any of 80%, 81%, 82%, 83%, 84%, 85%, 86%, 87%, 88%, 89%, 90%, 91%, 92%, 93%, 94%, 95%, 96%, 97%, 98%, 99%, or 100% identity thereto. In some embodiments, one or both of the two IgM heavy chain constant regions within an individual binding unit of an IgM antibody of the disclosure comprise the amino acid sequence of SEQ ID NO: 18, a fragment thereof, or an amino acid sequence with at least about any of 80%, 81%, 82%, 83%, 84%, 85%, 86%, 87%, 88%, 89%, 90%, 91%, 92%, 93%, 94%, 95%, 96%, 97%, 98%, 99%, or 100% identity thereto. In some embodiments, one or both of the two IgM heavy chain constant regions within an individual binding unit of an IgM antibody of the disclosure comprise a human IgM heavy chain constant region sequence (e.g., a wild type human IgM heavy chain constant region sequence), or fragments or variants thereof.

An anti-PSGL-1 IgM antibody of the disclosure, e.g., a pentameric antibody, can further comprise a J-chain, or functional fragment thereof, or a variant thereof. The J-chain may comprise a native J-chain sequence of IgM or IgA antibodies of any animal species, including mature human J-chain sequence. An exemplary J-chain sequence is provided herein as SEQ ID NO: 19.

In some embodiments, the J-chain is a modified J-chain. In some embodiments, the J-chain can comprise a heterologous moiety, e.g., a heterologous polypeptide, such as an extraneous binding domain, which can include, for example, a polypeptide binding domain capable of specifically binding to a target (e.g., to provide for bispecific or multi-specific IgM antibodies). The binding domain can be, for example, an antibody or antigen-binding fragment thereof, an antibody-drug conjugate or antigen-binding fragment thereof, an antibody-like molecule, a soluble or membrane bound protein, a ligand or a receptor. A polypeptide binding domain can be introduced into a J-chain by appropriately selecting the location and type of addition (e.g., direct or indirect fusion, chemical tethering, etc.). The binding domain can be introduced into the native J-chain sequence at any location that allows the binding of the binding domain to its binding target without interfering with the binding of a recipient IgM molecule to its binding target or binding targets or the ability of the J-chain to effectively incorporate into an IgM pentamer. In some embodiments, the binding domain can be inserted at or near the C-terminus, at or near the mature N-terminus or at an internal location that, based on the three-dimensional structure of the J-chain. In some embodiments, the binding domain can be introduced into a native sequence J-chain at or near a glycosylation site. The J-chain may also be modified to modulate physiological properties of the IgM antibody, such as to modulate (e.g., increase or decrease) clearance of the antibody from the circulation of a subject. For example, the J-chain may be modified to bind to albumin, albumin-like proteins, or albumin binding peptides, neonatal Fc receptor (FcRn). The J-chain sequence may be modified by direct or indirect fusion, i.e., by the combination of the J-chain and binding domain in one polypeptide chain by in-frame combination of their coding nucleotide sequences, with or without a peptide linker. The peptide linker (indirect fusion), if used, can be about 1 to 50, or about 1 to 40, or about 1 to 30, or about 1 to 20, or about 1 to 10, or about 10 to 20 amino acids in length, and can be present at one or both ends of the binding domain to be introduced into the J-chain sequence. Examples of modified J-chains, which may be used in the present disclosure, are provided, without limitation, in PCT Publication Nos. WO2015/153912, WO2017/059387, and WO2017/059380, which are herein incorporate by reference in their entirety.

J-chain polypeptides of the disclosure, including native sequence and modified J-chain polypeptides, can be produced by well-known techniques of recombinant DNA technology, by expressing a nucleic acid encoding the J-chain in a suitable prokaryotic or eukaryotic host organism, e.g., as described herein. The J-chains of the disclosure can also be co-expressed with the heavy and light chains, or fragments or variants thereof, of an IgM antibody as described herein.

In some embodiments, an anti-PSGL-1 IgM antibody of the disclosure comprises a J-chain comprising the amino acid sequence of SEQ ID NO: 19, or fragment or variant thereof, or an amino acid sequence having at least about any of 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 19.

An anti-PSGL-1 IgM antibody of the disclosure may be mono-specific, bispecific or multi-specific, e.g., a monospecific, bispecific, or multispecific human IgM antibody.

### Antibody Modifications and Variants

The present disclosure encompasses modifications to antibodies or fragments thereof described herein, including functionally equivalent antibodies which do not significantly affect their properties, and variants which have enhanced or decreased activity and/or affinity. Modification of polypeptides is routine practice in the art and need not be described in detail herein. Examples of modified polypeptides include polypeptides with conservative substitutions of amino acid residues, one or more deletions or additions of amino acids which do not significantly deleteriously change the functional activity, or use of chemical analogs.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to an epitope tag. Other insertional variants of the antibody molecule include the fusion to the N- or C-terminus of the antibody of an enzyme or a polypeptide which increases the serum half-life of the antibody.

Substitution variants have at least one amino acid residue in the antibody molecule removed and a different residue inserted in its place. The sites of greatest interest for substitutional mutagenesis include the hypervariable regions, but FR alterations are also contemplated. Conservative substitutions are shown in the table below under the heading of “conservative substitutions.” If such substitutions result in a change in biological activity, then more substantial changes, denominated “exemplary substitutions” in the table below, or as further described below in reference to amino acid classes, may be introduced and the products screened.

TABLE 2
Amino acid substitutions.

| Original | Conservative | Exemplary |
|---|---|---|
| Residue | Substitutions | Substitutions |
| Ala (A) | Val | Val; Leu; Ile |
| Arg (R) | Lys | Lys; Gln; Asn |
| Asn (N) | Gln | Gln; His; Asp, Lys; Arg |
| Asp (D) | Glu | Glu; Asn |
| Cys (C) | Ser | Ser; Ala |
| Gln (Q) | Asn | Asn; Glu |
| Glu (E) | Asp | Asp; Gln |
| Gly (G) | Ala | Ala |
| His (H) | Arg | Asn; Gln; Lys; Arg |
| Ile (I) | Leu | Leu; Val; Met; Ala; |
| | | Phe; Norleucine |
| Leu (L) | Ile | Norleucine; Ile; Val; |
| | | Met; Ala; Phe |
| Lys (K) | Arg | Arg; Gln; Asn |
| Met (M) | Leu | Leu; Phe; Ile |
| Phe (F) | Tyr | Leu; Val; Ile; Ala; Tyr |
| Pro (P) | Ala | Ala |
| Ser (S) | Thr | Thr |
| Thr (T) | Ser | Ser |
| Trp (W) | Tyr | Tyr; Phe |
| Tyr (Y) | Phe | Trp; Phe; Thr; Ser |
| Val (V) | Leu | Ile; Leu; Met; Phe; Ala; |
| | | Norleucine |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:
(1) Non-polar: Norleucine, Met, Ala, Val, Leu, Ile;
(2) Polar without charge: Cys, Ser, Thr, Asn, Gln;
(3) Acidic (negatively charged): Asp, Glu;
(4) Basic (positively charged): Lys, Arg;
(5) Residues that influence chain orientation: Gly, Pro; and
(6) Aromatic: Trp, Tyr, Phe, His.

Non-conservative substitutions are made by exchanging a member of one of these classes for another class.

Any cysteine residue not involved in maintaining the proper conformation of the antibody also may be substituted, generally with serine, to improve the oxidative stability of the molecule and prevent aberrant cross-linking. Conversely, cysteine bond(s) may be added to the antibody to improve its stability, particularly where the antibody is an antibody fragment such as an Fv fragment.

In some embodiments, an anti-PSGL-1 antibody of the present disclosure comprises an antibody constant domain, such as an IgM constant domain, e.g., as described herein. In some embodiments, the antibody constant domain is a human antibody constant domain. In some embodiments, one or more amino acid residues in the heavy chain constant region and/or the light chain constant region of the antibody are modified. For example, it may be desirable to modify an antibody of the disclosure with respect to effector function, e.g., so as to enhance or decrease antigen-dependent cell-mediated cytotoxicity (ADCC) and/or complement dependent cytotoxicity (CDC) of the antibody. See, e.g., Shields et al., J. Biol. Chem. 276:6591-6604 (2001); Presta et al., Biochem. Soc. Trans. 30:487-490 (2002). This may be achieved by introducing one or more amino acid substitutions in a heavy chain constant region of the antibody. In some embodiments, the anti-PSGL-1 antibodies of the disclosure may comprise an IgM constant region (e.g., a human IgM constant region) comprising one or more amino acid substitutions that reduce CDC activity of the antibody, for example, as compared to a corresponding wild-type IgM antibody. In some embodiments, the corresponding wild-type IgM antibody comprises a wild type IgM heavy chain constant region, such as an IgM human heavy chain constant region comprising the amino acid sequence of SEQ ID NO: 18. In some embodiments, the one or more amino acid substitutions that reduce CDC activity of the antibody are selected from the amino acid substitutions described in U.S. Pat. No. 11,401,337, which is hereby incorporated by reference in its entirety. The one or more amino acid substitutions that reduce CDC activity of the antibody may, in some cases, be within the Cμ3 domain of the antibody, which is thought to associate with the C1q component of complement. CDC activity of an antibody may be assessed using any suitable assay known in the art. For example, one method to assay CDC activity of an antibody is to mix target cells bound by the antibody being evaluated with an additive, e.g., serum, that contains the components of the complement system and then measure cell death. Cell death can be measured using any method known in the art, including radioactive and non-radioactive assays such as the CELLTITER-GLO® assay from Promega. CDC activity may also be assessed as described in Gazzano-Santoro et al., J. Immunol. Methods, 202:163 (1996).

Modifications to antibodies of the disclosure can also include post-translational modifications such as glycosylation, acetylation, and phosphorylation. Antibodies are glycosylated at conserved positions in their constant regions (Jefferis and Lund, 1997, Chem. Immunol. 65:111-128; Wright and Morrison, 1997, TibTECH 15:26-32). The oligosaccharide side chains of the immunoglobulins affect the protein's function (Boyd et al., 1996, Mol. Immunol. 32:1311-1318; Wittwe and Howard, 1990, Biochem. 29:4175-4180) and the intramolecular interaction between portions of the glycoprotein, which can affect the conformation and presented three-dimensional surface of the glycoprotein (Hefferis and Lund, supra; Wyss and Wagner, 1996, Current Opin. Biotech. 7:409-416). Oligosaccharides may also serve to target a given glycoprotein to certain molecules based upon specific recognition structures. Glycosylation of antibodies has also been reported to affect antibody-dependent cellular cytotoxicity (ADCC). In particular, CHO cells with tetracycline-regulated expression of P(1,4)-N-acetylglucosaminyltransferase III (GnTIII), a glycosyltransferase catalyzing formation of bisecting GlcNAc, was reported to have improved ADCC activity (Umana et al., 1999, Mature Biotech. 17:176-180).

Glycosylation of antibodies is typically either N-linked or O-linked. N-linked refers to the attachment of the carbohydrate moiety to the side chain of an asparagine residue. The tripeptide sequences asparagine-X-serine, asparagine-X-threonine, and asparagine-X-cysteine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used. Addition of glycosylation sites to an antibody of the disclosure is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

The glycosylation pattern of antibodies may also be altered without altering the underlying nucleotide sequence. Glycosylation largely depends on the host cell used to express the antibody. Since the cell type used for expression of recombinant glycoproteins, e.g., antibodies, as potential therapeutics is rarely the native cell, variations in the glycosylation pattern of the antibodies can be expected (see, e.g., Hse et al., 1997, J. Biol. Chem. 272:9062-9070).

In addition to the choice of host cells, factors that affect glycosylation during recombinant production of antibodies include growth mode, media formulation, culture density, oxygenation, pH, purification schemes, and the like. Various methods have been proposed to alter the glycosylation pattern achieved in a particular host organism including introducing or overexpressing certain enzymes involved in oligosaccharide production (U.S. Pat. Nos. 5,047,335; 5,510,261; and 5,278,299). Glycosylation, or certain types of glycosylation, can be enzymatically removed from the glycoprotein, for example using endoglycosidase H (Endo H), N-glycosidase F, endoglycosidase F1, endoglycosidase F2, or endoglycosidase F3. In addition, recombinant host cells can be genetically engineered to be defective in processing certain types of polysaccharides. These and similar techniques are well known in the art.

In some embodiments, an antibody of the present disclosure is modified using coupling techniques known in the art, including, but not limited to, enzymatic means, oxidative substitution, and chelation. Modifications can be used, for example, for attachment of labels for immunoassay.

An antibody of the present disclosure may be conjugated (for example, linked) to an agent, such as a therapeutic agent or a label. Examples of therapeutic agents are radioactive moieties, cytotoxins, and chemotherapeutic molecules. An antibody of the present disclosure may also be linked to a label such as a fluorescent molecule, a radioactive molecule, an enzyme, or any other labels known in the art. As used herein, the term “label” refers to any molecule that can be detected. In a certain embodiment, an antibody may be labeled by incorporation of a radiolabeled amino acid. In a certain embodiment, biotin moieties that can be detected by marked avidin (e.g., streptavidin containing a fluorescent marker or enzymatic activity that can be detected by optical or colorimetric methods) may be attached to the antibody. In certain embodiments, a label may be incorporated into or attached to another reagent which in turn binds to the antibody of interest. For example, a label may be incorporated into or attached to an antibody that in turn specifically binds the antibody of interest. In certain embodiments, the label or marker can also be therapeutic. Various methods of labeling polypeptides and glycoproteins are known in the art and may be used. Certain general classes of labels include, but are not limited to, enzymatic, fluorescent, chemiluminescent, and radioactive labels. Examples of labels for polypeptides include, but are not limited to, the following: radioisotopes or radionucleotides (e.g., ³H, ⁴C, ¹⁵N, ³⁵S, ⁹⁰Y ⁹⁹Tc, ¹¹¹In, ¹²⁵I, or ¹³¹I), fluorescent labels (e.g., fluorescein isothocyanate (FITC), rhodamine, lanthanide phosphors, or phycoerythrin (PE)), enzymatic labels (e.g., horseradish peroxidase, β-galactosidase, luciferase, alkaline phosphatase, glucose oxidase, glucose-6-phosphate dehydrogenase, alcohol dehydrogenase, malate dehydrogenase, penicillinase, or luciferase), chemiluminescent, biotinyl groups, predetermined polypeptide epitopes recognized by a secondary reporter (e.g., leucine zipper pair sequences, binding sites for secondary antibodies, metal binding domains, or epitope tags). In certain embodiments, labels are attached by spacer arms of various lengths to reduce potential steric hindrance.

### Antibody Activities and Assays

In some embodiments, an antibody of the present disclosure, e.g., an IgM anti-PSGL-1 antibody described herein, may be tested for induction of apoptosis in cell(s) expressing human PSGL-1. In some embodiments, an IgM antibody of the present disclosure displays enhanced induction of apoptosis in a target cell (e.g., a cell expressing human PSGL-1 or an epitope thereof, such as a T-cell, e.g., an activated T-cell) as compared to a control antibody or antibody fragment (e.g., a bivalent anti-PSGL-1 antibody or a non-specific antibody, or fragments thereof). Apoptosis assays are described in the art and can be readily carried out by one of skill in the art (see, e.g., Muppidi, J., Porter, M. and Siegel, R. M. 2004. Measurement of Apoptosis and Other Forms of Cell Death. Current Protocols in Immunology. 59:3.17.1-3.17.36). Exemplary and non-limiting methods for detecting apoptosis include, e.g., Annexin V or propidium iodide staining. In some embodiments, an antibody of the present disclosure may enhance induction of apoptosis in a target cell (e.g., a cell expressing human PSGL-1 or an epitope thereof, such as a T-cell, e.g., an activated T-cell) by about 5%, about 10%, about 20%, about 30%, about 40%, about 50%, about 60%, about 70%, about 80%, about 90%, about 100%, about 125%, about 150% or more, compared to cells not treated with the antibody, or to cells treated with a control antibody or antibody fragment (e.g., a bivalent or tetravalent anti-PSGL-1 antibody or a non-specific antibody, or fragments thereof).

In some embodiments, a control antibody or antibody fragment suitable for use according to the present disclosure is an antibody (or antibody fragment) that binds to PSGL-1, such as human PSGL-1, e.g., a monovalent or bivalent antibody or antibody fragment. In some embodiments, the control antibody is an anti-PSGL-1 antibody (or antibody fragment) described in, e.g., U.S. Pat. No. 7,604,800; WO2012/174001; Constantin, Gabriela, 2004, Drug News Perspect., 17(9): 579-585; Chen et al., Cross-Linking of P-Selectin Glycoprotein Ligand-1 Induces Death of Activated T-cells, Blood 104(10):3233-3242; and Huang et al., A Novel Apoptosis-Inducing Anti-PSGL-1 Antibody for T-cell-Mediated Diseases, Eur. J. Immunol. 2005, 35(7):2239-49). In some embodiments, the control antibody (e.g., an anti-PSGL-1 antibody) comprises a light chain comprising the amino acid sequence of SEQ ID NO:23 and a heavy chain comprising the amino acid sequence of SEQ ID NO:24. In some embodiments, one or two Fc domains of the control antibody do not have the C-terminal lysine residue.

An antibody of the present disclosure may be also tested according to a variety of other in vitro and in vivo assays known in the art. Such assays may include, e.g., binding assays directed to the ability of an antibody or fragment thereof to bind an epitope or polypeptide of interest (e.g., human PSGL-1 or an epitope thereof), or functional assays directed to one or more functional properties of an antibody or fragment thereof, e.g., as described above. In some embodiments, an antibody of the present disclosure may be tested for binding activity against human PSGL-1. In some embodiments, binding of an antibody to human PSGL-1 or an epitope thereof may be tested in an in vitro binding assay. In some embodiments, an antibody of the present disclosure may be tested for its ability to interfere with binding of PSGL-1 to one or more PSGL-1 ligands, such as P-Selectin, L-Selectin, or E-Selectin. In some embodiments, an antibody of the disclosure does not interfere with binding between PSGL-1 and P-Selectin, L-Selectin, and/or E-Selectin. In some embodiments, binding between PSGL-1 and P-Selectin, L-Selectin, or E-Selectin, e.g., in the presence or absence of an antibody of the disclosure, may be tested in an in vitro binding assay. A variety of binding assays are known in the art. Such binding assays may be cell-based assays (e.g., testing the ability of an antibody, or a PSGL-1 ligand such as P-Selectin, L-Selectin, or E-Selectin, to bind a cell expressing human PSGL-1 or an epitope thereof), or they may be polypeptide-based (e.g., testing the ability of an antibody, or a PSGL-1 ligand such as P-Selectin, L-Selectin, or E-Selectin, to bind human PSGL-1 or an epitope thereof). In some embodiments, an antibody of the present disclosure may be tested for binding to a cell expressing human PSGL-1, e.g., by flow cytometry, FRET, histochemical assays, and the like. Other suitable binding assays may include, without limitation, equilibrium methods, e.g., enzyme-linked immunosorbent assay (ELISA), radioimmunoassay (RIA), Biacore™ analysis, indirect binding assay, competitive inhibition assay, fluorescence resonance energy transfer (FRET), immunoprecipitation, gel electrophoresis and chromatography (e.g., gel filtration).

### III. Nucleic Acids, Vectors, Host Cells, and Antibody Production

The present disclosure also provides nucleic acid(s) (e.g., polynucleotides) encoding any of the antibodies described herein, or portions thereof. In some embodiments, the nucleic acids encode the sequences of light chain and/or heavy chain variable regions of any of the antibodies described herein, or portions or variants thereof. In other embodiments, the nucleic acids encode the sequences of a constant region (e.g., a heavy or light chain constant region) of any of the antibodies described herein, or portions or variants thereof. In other embodiments, the nucleic acids encode the sequence of a J-chain polypeptide described herein, or portions or variants thereof. In other embodiments, the nucleic acids encode the sequences of light chains and/or heavy chains of any of the antibodies described herein, or portions or variants thereof. In some embodiments, the nucleic acids are isolated (e.g., isolated from a host cell or from one or more different nucleic acids).

In some embodiments, provided herein are nucleic acids encoding any of the antibodies described herein, or portions or variants thereof (e.g., monomers such as single-chain polypeptides, antibody fragments, antibody heavy chain variable regions, antibody light chain variable regions, J-chain polypeptides, tailpiece polypeptides, antibody constant regions, antibody heavy chains, and/or antibody light chains). In some embodiments, a nucleic acid of the present disclosure encodes a polypeptide sequence selected from SEQ ID NOs:1-20, and any combination thereof. In some embodiments, a nucleic acid of the present disclosure comprises a polynucleotide sequence encoding any of SEQ ID NOs: 15, 16, 17, 18, 19, and 20, and any combination thereof. In some embodiments, a nucleic acid of the present disclosure comprises one or more introns. In other embodiments, a nucleic acid of the present disclosure does not comprise an intron, e.g., a cDNA or processed mRNA sequence.

It is appreciated by those of ordinary skill in the art that, as a result of the degeneracy of the genetic code, there are many nucleotide sequences that encode a polypeptide as described herein. Some of these nucleic acids bear minimal homology to the nucleotide sequence of any native gene or reference sequence. Thus, nucleic acids that vary due to differences in codon usage are specifically contemplated by the present disclosure. Further, alleles of the polynucleotide sequences provided herein are within the scope of the present disclosure. Alleles are endogenous genes that are altered as a result of one or more mutations, such as deletions, additions, and/or substitutions of nucleotides. The resulting mRNA and protein can, but need not, have an altered structure or function. Alleles can be identified using standard techniques (such as hybridization, amplification, and/or database sequence comparison).

Also provided herein are nucleic acids (e.g., polynucleotides) that are optimized, e.g., by codon/RNA optimization, replacement with heterologous signal sequences, and/or elimination of mRNA instability elements. Methods to generate optimized nucleic acids encoding an antibody, or a fragment or variant thereof, for recombinant expression by introducing codon changes and/or eliminating inhibitory regions in the mRNA can be carried out by adapting the optimization methods described in, e.g., U.S. Pat. Nos. 5,965,726; 6, 174,666; 6,291,664; 6,414, 132; and 6,794,498, accordingly. For example, potential splice sites and instability elements (e.g., A/T or A/U rich elements) within the RNA can be mutated without altering the amino acids encoded by the nucleic acid sequences to increase stability of the RNA for recombinant expression. The alterations utilize the degeneracy of the genetic code, e.g., using an alternative codon for an identical amino acid. In some embodiments, it can be desirable to alter one or more codons to encode a conservative mutation, e.g., a similar amino acid with similar chemical structure and properties and/or function as the original amino acid. Such methods can increase expression of an anti-PSGL-1 antibody, or a fragment or variant thereof, relative to the expression of an anti-PSGL-1 antibody, or a fragment or variant thereof, encoded by polynucleotides that have not been optimized. Furthermore, the polynucleotide sequences can be designed to match the preferred codon usage in a host cell, e.g., E. coli codon usage or CHO codon usage.

An optimized polynucleotide sequence encoding an antibody, or a fragment or variant thereof, described herein can hybridize to an unoptimized polynucleotide sequence encoding an antibody, or a fragment or variant thereof, described herein. In specific embodiments, an optimized nucleotide sequence encoding an antibody, or a fragment or variant thereof, described herein hybridizes under high stringency conditions to an unoptimized polynucleotide sequence encoding an antibody, or a fragment or variant thereof, described herein. In a specific embodiment, an optimized nucleotide sequence encoding an antibody, or a fragment or variant thereof, described herein hybridizes under high stringency, intermediate or lower stringency hybridization conditions to an unoptimized nucleotide sequence encoding an antibody, or a fragment or variant thereof, described herein. Information regarding hybridization conditions have been described, see, e.g., U.S. Patent Application Publication No. US 2005/0048549 (e.g., paragraphs 72-73), which is incorporated herein by reference in its entirety.

The nucleic acids of the present disclosure can be obtained using chemical synthesis, recombinant methods, or PCR. Methods of chemical polynucleotide synthesis are well known in the art and need not be described in detail herein. One of skill in the art can use the sequences provided herein and a commercial DNA synthesizer to produce a desired DNA sequence.

For preparing nucleic acids (e.g., polynucleotides) using recombinant methods, a polynucleotide comprising a desired sequence can be inserted into a suitable vector, and the vector in turn can be introduced into a suitable host cell for replication and amplification, as further discussed herein. Nucleic acids can be inserted into host cells by any means known in the art. Cells are transformed by introducing an exogenous polynucleotide by direct uptake, endocytosis, transfection, F-mating, or electroporation. Once introduced, the exogenous polynucleotide can be maintained within the cell as a non-integrated vector (such as a plasmid) or integrated into the host cell genome. The polynucleotide so amplified can be isolated from the host cell by methods well known within the art. See, e.g., Sambrook et al. (1989).

Alternatively, PCR allows reproduction of DNA sequences. PCR technology is well known in the art and is described in U.S. Pat. Nos. 4,683,195; 4,800,159; 4,754,065; and 4,683,202, as well as PCR: The Polymerase Chain Reaction, Mullis et al. eds., Birkauswer Press, Boston (1994).

The present disclosure also provides vectors (e.g., cloning vectors or expression vectors) comprising a nucleic acid sequence encoding any of the polypeptides (including antibodies, or fragments or variants thereof,) described herein. Suitable cloning vectors can be constructed according to standard techniques or may be selected from a large number of cloning vectors available in the art. While the cloning vector selected may vary according to the host cell intended to be used, useful cloning vectors generally have the ability to self-replicate, may possess a single target for a particular restriction endonuclease, and/or may carry genes for a marker that can be used in selecting clones containing the vector. Suitable examples include plasmids and bacterial viruses, e.g., pUC18, pUC19, Bluescript (e.g., pBS SK+) and its derivatives, mp18, mp19, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

Expression vectors generally are replicable polynucleotide constructs that contain a nucleic acid according to the present disclosure. The expression vector may be replicable in host cells either as episomes or as an integral part of chromosomal DNA. Suitable expression vectors include but are not limited to plasmids, viral vectors, including adenoviruses, adeno-associated viruses, retroviruses, cosmids, and expression vector(s) disclosed in PCT Publication No. WO 87/04462. Vector components may generally include, but are not limited to, one or more of the following: a signal sequence; an origin of replication; one or more marker genes; and suitable transcriptional controlling elements (such as promoters, enhancers, or terminator). For expression (i.e., translation), one or more translational controlling elements are also usually required, such as ribosome binding sites, translation initiation sites, or stop codons.

Methods of making antibodies, or fragments or variants thereof, and polypeptides derived from the antibodies are known in the art and are disclosed herein. Well-established methods may be used to identify anti-PSGL-1 antibodies (e.g., antibodies that specifically bind to human PSGL-1), from which variable domains (e.g., VH and/or VL domains) may be used in the antibodies of the present disclosure. Exemplary anti-human PSGL-1 antibodies, as well as methods for screening, producing, and purifying such antibodies, are described in International Application Pub. No. WO 2012/174001.

Additional anti-human PSGL-1 antibodies may be identified using methods known in the art, such as those described in International Application Pub. No. WO 2012/174001 and supra. For example, monoclonal antibodies can be prepared using hybridoma technology, such as those described by Kohler and Milstein (1975), Nature, 256:495. In a hybridoma method, a mouse, a hamster, or other appropriate host animal, is typically immunized with an immunizing agent (e.g., a cell expressing human PSGL-1 or a fragment thereof) to elicit lymphocytes that produce or are capable of producing antibodies that specifically bind to the immunizing agent. Alternatively, the lymphocytes may be immunized in vitro. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-1031). Immortalized cell lines are usually transformed mammalian cells, particularly myeloma cells of rodent, rabbit, bovine, or human origin. Usually, rat or mouse myeloma cell lines are employed. The hybridoma cells may be cultured in a suitable culture medium that desirably contains one or more substances that inhibit the growth or survival of unfused, immortalized cells. For example, if the parental cells lack the enzyme hypoxanthine guanine phosphoribosyl transferase (HGPRT or HPRT), the culture medium for the hybridomas typically includes hypoxanthine, aminopterin, and thymidine (“HAT medium”), which substances prevent the growth of HGPRT-deficient cells.

Desired immortalized cell lines are those that fuse efficiently, support stable high level expression of antibody by the selected antibody-producing cells, and are sensitive to a medium such as HAT medium. More desirable immortalized cell lines are murine myeloma lines, which can be obtained, for instance, from the Salk Institute Cell Distribution Center, San Diego, CA and the American Type Culture Collection, Manassas, VA. Human myeloma and mouse-human heteromyeloma cell lines also have been described for the production of human monoclonal antibodies (Kozbor, J. Immunol. (1984), 133:3001; Brodeur et al., Monoclonal Antibody Production Techniques and Applications, Marcel Dekker, Inc., New York, (1987) pp. 51-63).

The culture medium in which hybridoma cells are cultured can then be assayed for the presence of antibodies, e.g., monoclonal antibodies. The antibody may be screened for having specific binding to a PSGL-1 polypeptide. A polypeptide comprising the sequence of SEQ ID NOs: 21 or 22, or fragments or variants thereof, may also be used for screening.

In some embodiments, the binding specificity of antibodies produced by the hybridoma cells is determined by immunoprecipitation or by an in vitro binding assay, such as radioimmunoassay (RIA) or enzyme-linked immunosorbent assay (ELISA). Such techniques and assays are known in the art. The binding affinity of an antibody can, for example, be determined by the Scatchard analysis of Munson and Pollard (1980), Anal. Biochem., 107:220.

After the desired hybridoma cells are identified, the clones may be subcloned by limiting dilution procedures and grown by standard methods (Goding, supra). Suitable culture media for this purpose include, for example, Dulbecco's Modified Eagle's Medium or RPMI-1640 medium. Alternatively, the hybridoma cells may be grown in vivo as ascites in a mammal.

The antibodies can be generated by culturing the hybridoma cells, and the antibodies secreted by the hybridoma cells may further be isolated or purified. Antibodies may be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The antibodies, or a fragment or variant thereof, of the present disclosure may be generated by screening a library of antibodies or polypeptides to select antibodies or polypeptides that bind to human PSGL-1, e.g., expressed on the cell surface of a cell. Antibody phage display libraries known in the art may be used. In some embodiments, the antibodies in the library (e.g., displayed on phage) are single-chain Fv (scFv) fragments or Fab fragment. In some embodiments, the antibodies in the library (e.g., displayed on phage) are single-domain antibodies. For example, a single-domain antibody may comprise all or a portion of the heavy chain variable domain or all or a portion of the light chain variable domain of an antibody. In some embodiments, the antibodies in the library are human antibodies. The antibodies identified may further be tested for their capabilities to induce cell death (e.g., apoptosis) and/or bind human PSGL-1 using methods known in the art and described herein.

The antibodies of the present disclosure can be made by recombinant DNA methods, such as those described in U.S. Pat. Nos. 4,816,567 and 6,331,415. For example, DNA encoding the variable or constant region of any of the antibodies of the present disclosure (or single, heavy, or light chain polypeptides that are constituents thereof) can be readily isolated and sequenced using conventional procedures (e.g., by using oligonucleotide probes that are capable of binding specifically to genes encoding the heavy and light chains of antibodies). The hybridoma cells of the present disclosure serve as a possible source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein to synthesize monoclonal antibodies in the recombinant host cells. The DNA also can be modified, for example, by substituting the coding sequence for human heavy and light chain constant domains in place of homologous murine sequences (U.S. Pat. No. 4,816,567) or by covalently joining to the immunoglobulin coding sequence to all or part of the coding sequence for a non-immunoglobulin polypeptide. Such a non-immunoglobulin polypeptide can be substituted for the constant domains of an antibody of the present disclosure, or can be substituted for the variable domains of one antigen-combining site of an antibody of the present disclosure to create a chimeric bivalent antibody.

In some embodiments, the antibodies of the present disclosure are expressed from two or more expression vectors. For example, each expression vector may express one monomer of a dimer or multimer (e.g., pentamer or hexamer) of the present disclosure (e.g., a single-chain polypeptide or antibody heavy or light chain polypeptide). Alternatively, more than one monomer of a dimer or multimer (e.g., pentamer or hexamer) of the present disclosure is expressed from a single expression vector.

Normally the expression vector has transcriptional and translational regulatory sequences which are derived from a species compatible with a host cell. In addition, the vector ordinarily carries a specific gene(s) which is (are) capable of providing phenotypic selection in transformed cells.

A wide variety of recombinant host-vector expression systems for eukaryotic cells are known and can be used in the present disclosure. For example, Saccharomyces cerevisiae, or common baker's yeast, is the most commonly used among eukaryotic microorganisms, although a number of other strains, such as Pichia pastoris, are available. Cell lines derived from multicellular organisms such as Sp2/0 or Chinese Hamster Ovary (CHO), which are available from the ATCC, may also be used as hosts. Typical vector plasmids suitable for eukaryotic cell transformations are, for example, pSV2neo and pSV2gpt (ATCC), pSVL and pSVK3 (Pharmacia), and pBPV-1/pML2d (International Biotechnology, Inc.).

Eukaryotic host cells useful in the present disclosure are, for example, hybridoma, myeloma, plasmacytoma, or lymphoma cells. However, other eukaryotic host cells, such as mammalian cells, may be suitably utilized provided the host cells are capable of recognizing transcriptional and translational DNA sequences for expression of the proteins; processing the leader peptide by cleavage of the leader sequence and secretion of the proteins; and providing post-translational modifications of the proteins, e.g., glycosylation.

Accordingly, the present disclosure provides host cells (e.g., eukaryotic host cells) which are transformed by recombinant expression vectors comprising DNA constructs disclosed herein and which are capable of expressing the antibodies, or fragments or variants thereof, of the present disclosure. In some embodiments, the transformed host cells of the present disclosure comprise at least one DNA construct comprising a nucleic acid of the present disclosure, or a nucleic acid encoding an antibody, or fragments or variants thereof, of the present disclosure (e.g., a monomer of an antibody of the present disclosure), and transcriptional and translational regulatory sequences which are positioned in relation to the coding DNA sequences to direct expression of antibodies, or fragments or variants thereof.

Any host cells capable of over-expressing heterologous DNAs can be used for the purpose of isolating genes encoding an antibody, polypeptide, or protein of interest. Non-limiting examples of mammalian host cells include but not limited to COS, HeLa, and CHO cells. See also PCT Publication No. WO 87/04462. Suitable non-mammalian host cells include prokaryotes (such as E. coli or B. subtillis) and yeast (such as S. cerevisae, S. pombe, or K. lactis).

The host cells used in the present disclosure may be transformed in a variety of ways by standard transfection procedures well known in the art. Among the standard transfection procedures which may be used are electroporation techniques, protoplast fusion and calcium-phosphate precipitation techniques. Such techniques are generally described by F. Toneguzzo et al. (1986), Mol. Cell. Biol., 6:703-706; G. Chu et al., Nucleic Acid Res. (1987), 15:1311-1325; D. Rice et al., Proc. Natl. Acad. Sci. USA (1979), 79:7862-7865; and V. Oi et al., Proc. Natl. Acad. Sci. USA (1983), 80:825-829. Vectors containing polynucleotides of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides often depends on features of the host cell.

In the case of two or more expression vectors, the two or more expression vectors can be transferred into a host cell one by one separately or together (co-transfer or co-transfect).

The present disclosure also provides a method for producing the antibodies, or fragments or variants thereof, that comprises culturing a host cell comprising an expression vector(s) encoding the antibodies, or fragments or variants thereof, and recovering the antibodies or polypeptides from the culture by ways well known to one skilled in the art.

Furthermore, the desired antibodies can be produced in a transgenic animal. A suitable transgenic animal can be obtained according to standard methods which include micro-injecting into eggs the appropriate expression vectors, transferring the eggs into pseudo-pregnant females, and selecting a descendant expressing the desired antibody.

The present disclosure also provides chimeric antibodies that specifically bind human PSGL-1. For example, the variable and constant regions of the antibody may be from separate species. In some embodiments, the variable regions of heavy chains and/or light chains (or portions thereof, such as the CDRs) are from murine antibodies, and optionally the constant regions or portions thereof, and/or framework regions, or portions thereof, of the antibody are from human antibodies. Chimeric antibodies of the present disclosure can be prepared by techniques well-established in the art. See for example, U.S. Pat. Nos. 6,808,901; 6,652,852; 6,329,508; 6,120,767; and 5,677,427, each of which is hereby incorporated by reference. In general, the chimeric antibody can be prepared by obtaining cDNAs encoding the heavy and light chain variable regions of the antibodies, inserting the cDNAs into an expression vector, which upon being introduced into eukaryotic host cells, expresses the chimeric antibody of the present disclosure. Desirably, the expression vector carries a functionally complete constant heavy or light chain sequence so that any variable heavy or light chain sequence can be easily inserted into the expression vector.

The present disclosure provides humanized antibodies, or fragments or variants thereof, that specifically bind to human PSGL-1. The humanized antibodies are typically human antibodies in which residues from CDRs are replaced with residues from CDRs of a non-human species such as mouse, rat, or rabbit having the desired specificity, affinity and capacity. In some instances, Fv framework residues of the human antibody are replaced by corresponding non-human residues.

There are four general steps to humanize a monoclonal antibody. These are: (1) determining the nucleotide and predicted amino acid sequence of the starting antibody light and heavy variable domains, (2) designing the humanized antibody, i.e., deciding which antibody framework region to use during the humanizing process, (3) the actual humanizing methodologies/techniques, and (4) the transfection and expression of the humanized antibody. See, for example, U.S. Pat. Nos. 4,816,567; 5,807,715; 5,866,692; 6,331,415; 5,530,101; 5,693,761; 5,693,762; 5,585,089; 6,180,370; and 6,548,640. For example, the constant region may be engineered to more resemble human constant regions to avoid immune response if the antibody is used in clinical trials and treatments in humans. See, for example, U.S. Pat. Nos. 5,997,867 and 5,866,692.

It is important that antibodies be humanized with retention of high affinity for the antigen and other favorable biological properties. To achieve this goal, humanized antibodies can be prepared by a process of analysis of the parental sequences and various conceptual humanized products using three dimensional models of the parental and humanized sequences. Three dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequence so that the desired antibody characteristic, such as increased affinity for the target antigen(s), is achieved. In general, the CDR residues are directly and most substantially involved in influencing antigen binding. The humanized antibodies may also contain modifications in the hinge region to improve one or more characteristics of the antibody.

In another alternative, antibodies may be screened and made recombinantly by phage display technology. See, for example, U.S. Pat. Nos. 5,565,332; 5,580,717; 5,733,743 and 6,265,150; and Winter et al., Annu. Rev. Immunol. 12:433-455 (1994). Alternatively, the phage display technology (McCafferty et al., Nature 348:552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats; for a review see, e.g., Johnson, Kevin S. and Chiswell, David J., Current Opinion in Structural Biology 3, 564-571 (1993). Several sources of V-gene segments can be used for phage display. Clackson et al., Nature 352:624-628 (1991) isolated a diverse array of anti-oxazolone antibodies from a small random combinatorial library of V genes derived from the spleens of immunized mice. A repertoire of V genes from unimmunized human donors can be constructed, and antibodies to a diverse array of antigens (including self-antigens) can be isolated essentially following the techniques described by Mark et al., J. Mol. Biol. 222:581-597 (1991), or Griffith et al., EMBO J. 12:725-734 (1993). In a natural immune response, antibody genes accumulate mutations at a high rate (somatic hypermutation). Some of the changes introduced will confer higher affinity, and B-cells displaying high-affinity surface immunoglobulin are preferentially replicated and differentiated during subsequent antigen challenge. This natural process can be mimicked by employing the technique known as “chain shuffling.” Marks et al., Bio Technol. 10:779-783 (1992)). In this method, the affinity of “primary” human antibodies obtained by phage display can be improved by sequentially replacing the heavy and light chain V region genes with repertoires of naturally occurring variants (repertoires) of V domain genes obtained from unimmunized donors. This technique allows the production of antibodies and antibody fragments with affinities in the pM-nM range. A strategy for making very large phage antibody repertoires (also known as “the mother-of-all libraries”) has been described by Waterhouse et al., Nucl. Acids Res. 21:2265-2266 (1993). Gene shuffling can also be used to derive human antibodies from rodent antibodies, where the human antibody has similar affinities and specificities to the starting rodent antibody. According to this method, which is also referred to as “epitope imprinting,” the heavy or light chain V domain gene of rodent antibodies obtained by phage display technique is replaced with a repertoire of human V domain genes, creating rodent-human chimeras. Selection on antigen results in isolation of human variable regions capable of restoring a functional antigen-binding site, i.e., the epitope governs (imprints) the choice of partner. When the process is repeated in order to replace the remaining rodent V domain, a human antibody is obtained (see PCT Publication No. WO 93/06213, published Apr. 1, 1993). Unlike traditional humanization of rodent antibodies by CDR grafting, this technique provides completely human antibodies, which have no framework or CDR residues of rodent origin. It is apparent that although the above discussion pertains to humanized antibodies, the general principles discussed are applicable to customizing antibodies for use, for example, in dogs, cats, primates, equines, and bovines.

In certain embodiments, an antibody, or a fragment or variant thereof, of the disclosure is a fully human antibody. Non-human antibodies that specifically bind an antigen can be used to produce a fully human antibody that binds to that antigen. For example, the skilled artisan can employ a chain swapping technique, in which the heavy chain of a non-human antibody is co-expressed with an expression library expressing different human light chains. The resulting hybrid antibodies, containing one human light chain and one non-human heavy chain, are then screened for antigen binding. The light chains that participate in antigen binding are then co-expressed with a library of human antibody heavy chains. The resulting human antibodies are screened once more for antigen binding. Techniques such as this one are further described in U.S. Pat. No. 5,565,332. In addition, an antigen can be used to inoculate an animal that is transgenic for human immunoglobulin genes. See, e.g., U.S. Pat. No. 5,661,016.

The present disclosure also provides bispecific or multi-specific antibodies. A bispecific or multi-specific antibody has binding specificities for at least two different antigens (including different epitopes). In some embodiments, a bispecific or multi-specific antibody of the present disclosure includes two or more different VH and/or VL domains that specifically bind PSGL-1. In some embodiments, the two or more different VH and/or VL domains specifically bind the same epitope of PSGL-1. In some embodiments, the two or more different VH and/or VL domains specifically bind different epitopes of PSGL-1, which may or may not be overlapping epitopes.

A bispecific or multi-specific antibody (an antibody that has binding specificities for at least two different antigens) can be prepared using the antibodies disclosed herein. Methods for making bispecific or multi-specific antibodies are known in the art (see, e.g., Suresh et al., 1986, Methods in Enzymology 121:210). Traditionally, the recombinant production of bispecific antibodies was based on the coexpression of two immunoglobulin heavy chain-light chain pairs, with the two heavy chains having different specificities (Millstein and Cuello, 1983, Nature 305, 537-539). In some embodiments, a bispecific antibody may be produced using the methods exemplified herein.

According to one approach to making bispecific or multi-specific antibodies, antibody variable domains with the desired binding specificities (antibody-antigen combining sites) are fused to immunoglobulin constant domain sequences. DNAs encoding the immunoglobulin heavy chain fusions and, if desired, the immunoglobulin light chain, are inserted into separate expression vectors, and are co-transfected into a suitable host organism or cell.

Heteroconjugate antibodies, comprising two or more covalently joined monomers or antibodies, are also within the scope of the present disclosure. Such antibodies have been used to target immune system cells to unwanted cells (U.S. Pat. No. 4,676,980), and for treatment of HIV infection (PCT Publication Nos. WO 91/00360 and WO 92/200373; and EP 03089). Heteroconjugate antibodies may be made using any convenient cross-linking methods. Suitable cross-linking agents and techniques are well known in the art, and are described in U.S. Pat. No. 4,676,980.

Certain aspects of the present disclosure relate to antibody variable domains and/or antibody fragments, e.g., that may be used as a constituent of an antibody described herein. Antibody fragments may contain the active binding region of the antibodies, such as Fab, F(ab′)2, scFv, Fv fragments, and the like. Various methods known in the art may be used to produce and/or isolate antibody fragments, which may be incorporated into an antibody of the present disclosure, e.g., by standard recombinant techniques known in the art based on the concepts described herein.

Single-chain Fv fragments may be produced, such as described in Iliades et al., 1997, FEBS Letters, 409:437-441. Coupling of such single-chain fragments using various linkers is described in Kortt et al., 1997, Protein Engineering, 10:423-433. A variety of techniques for the recombinant production and manipulation of antibodies are well known in the art. Such fragments can be produced from the monoclonal antibodies described herein using techniques well established in the art (Rousseaux et al. (1986), in Methods Enzymol., 121:663-69 Academic Press).

Methods of preparing antibody fragments are well known in the art. For example, an antibody fragment can be produced by enzymatic cleavage of antibodies with pepsin to provide a 100 Kd fragment denoted F(ab′)₂. This fragment can be further cleaved using a thiol reducing agent, and optionally a blocking group for the sulfhydryl groups resulting from cleavage of disulfide linkages, to produce 50 Kd Fab′ monovalent fragments. Alternatively, an enzymatic cleavage using papain produces two monovalent Fab fragments and an Fc fragment directly. These methods are described, for example, by U.S. Pat. Nos. 4,036,945 and 4,331,647 and references contained therein, which patents are incorporated herein by reference. Also, see Nisonoff et al. (1960), Arch Biochem. Biophys. 89: 230; Porter (1959), Biochem. J. 73: 119; Smyth (1967), Methods in Enzymology 11: 421-426. Alternatively, the Fab can be produced by inserting DNA encoding Fab of the antibody into an expression vector for a prokaryote or an expression vector for a eukaryote, and introducing the vector into a prokaryote or eukaryote to express the Fab.

### IV. Methods and Uses

Certain aspects of the present disclosure relate to methods and uses for the anti-PSGL-1 IgM antibodies described herein. These methods and uses are based, at least in part, on the properties of the antibodies as described herein, including without limitation, their increased number of epitope binding domains, and increased potency and efficacy for downregulation of activated T cells.

PSGL-1 is known to be involved in inflammation and T-cell biology. The antibodies of the present disclosure that specifically bind human PSGL-1 may find use, inter alia, in treating individuals with diseases related to T-cell function (e.g., a T-cell mediated inflammatory disease or a cancer), or individuals in need of medical procedures that may result in inflammatory conditions such as immunological reactions, or for which such conditions are managed beforehand (e.g., a transplantation or transfusion).

In some embodiments, a disorder or disease treated by the methods described herein may be a T-cell mediated inflammatory disease. Non-limiting examples of disorders and diseases that can be treated, or one or more of whose symptoms may be ameliorated or prevented using the antibodies described herein include psoriasis, Crohn's disease, ankylosing spondylitis, arthritis (including rheumatoid arthritis, juvenile rheumatoid arthritis, osteoarthritis, and psoriatic arthritis), diabetes mellitus (including type I diabetes), multiple sclerosis, encephalomyelitis, myasthenia gravis, lupus, systemic lupus erythematosus, autoimmune thyroiditis, autoimmune thyroid disorder, dermatitis (including atopic dermatitis and eczematous dermatitis), Sjogren's Syndrome, aphthous ulcer, iritis, conjunctivitis, keratoconjunctivitis, type I diabetes, inflammatory bowel diseases, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, graft versus host disease (GVHD), sarcoidosis, primary biliary cirrhosis, uveitis posterior, interstitial lung fibrosis, allergies such as atopic allergy, AIDS, immunoglobulin nephropathies, a skin disorder, myocarditis, T-cell neoplasms such as leukemias or lymphomas, psoriatic arthritis, vitiligo, alopecia areata, Steven Johnson Syndrome (SJS), Drug reaction with eosinophilia and systemic symptoms (Dress), T-cell mediated skin diseases, and juvenile arthritis. In some embodiments, the disease or disorder is an autoimmune disease or disorder.

In some embodiments, the disease or disorder treated in accordance with the methods described herein is plaque psoriasis. Plaque psoriasis or psoriasis vulgaris is the most common form of psoriasis and is characterized by sharply demarcated, raised erythematous skin plaques covered by silvery scale. There is a predilection of the lesions to involve the extensor surfaces of the extremities, the lumbosacral area, and the scalp. The corresponding histopathological findings include significant inflammatory cellular infiltration of the dermis and epidermis, increased numbers of dilated vessels, and a substantial thickening of the epidermis with disordered differentiation of keratinocytes and hyperkeratosis. Approximately one third of patients with plaque psoriasis are categorized as having moderate or severe disease and are consequently candidates for therapy beyond just topical treatment. In some embodiments, the disorder treated in accordance with the methods described herein is chronic plaque psoriasis. Symptoms of chronic plaque psoriasis include, but are not limited to, single or multiple raised reddened patches of skin, ranging from coin-sized to larger, on any part of the body, including but not limited to the knees, elbows, lumbosacral regions, scalp, and nails. In some embodiments, the disorder to be treated in accordance with the methods described herein is guttate psoriasis. Symptoms of guttate psoriasis include, but are not limited to, flares of water drop shaped scaly plaques on the skin, followed by an infection, such as a streptococcal throat infection. In some embodiments, the disease or disorder to be treated in accordance with the methods described herein is inverse psoriasis. Symptoms of inverse psoriasis include, but are not limited to, smooth, usually moist areas of skin that are red and inflamed, unlike the scaling associated with plaque psoriasis, on one or more of the following body parts: armpits, groin, under the breasts, and in other skin folds around the genitals and buttocks. In some embodiments, the disease or disorder to be treated in accordance with the methods described herein is pustular psoriasis. Symptoms of pustular psoriasis include, but are not limited to, pus-filled blisters that vary in size and location, but mostly on the hands and feet. In some embodiments, the disease or disorder to be treated in accordance with the methods described herein is erythodermic psoriasis. Symptoms of erythodermic psoriasis include, but are not limited to, periodic, widespread, fiery redness of the skin and the shedding of scales in sheets, rather than smaller flakes. The reddening and shedding of the skin are often accompanied by severe itching and pain, heart rate increase, and fluctuating body temperature.

In some embodiments, the disease or disorder to be treated in accordance with the methods described herein is rheumatoid arthritis. Symptoms of rheumatoid arthritis, include, but are not limited to, fatigue, loss of appetite, low fever, swollen glands, weakness, joint pain in wrists, elbows, shoulders, hips, knees, ankles, toes, jaw, hands, feet, fingers, and/or neck, morning stiffness, chest pain when taking a breath (pleurisy), eye burning, itching, and discharge, nodules under the skin, numbness, tingling, or burning in the hands and feet.

In some embodiments, the disease or disorder to be treated in accordance with the methods described herein is Crohn's disease. Symptoms of Crohn's disease include, but are not limited to, crampy abdominal (belly area) pain, fever, fatigue, loss of appetite, pain with passing stool (tenesmus), persistent, watery diarrhea, unintentional weight loss, constipation, eye inflammation, fistulas (usually around the rectal area, may cause draining of pus, mucus, or stools), joint pain, liver inflammation, mouth ulcers, rectal bleeding and bloody stools, skin lumps or sores (ulcers), and swollen gums.

In some embodiments, the disease or disorder to be treated in accordance with the methods described herein is ankylosing spondylitis. Symptoms of ankylosing spondylitis include, but are not limited to, frequent pain and stiffness in the lower back and buttocks, spine, and/or neck; and pain and tenderness spreading to the ribs, shoulder blades, hips, thighs and heels; inflammation of the eye (iridocyclitis and uveitis), causing redness, eye pain, vision loss, floaters and photophobia; fatigue; and nausea.

In some embodiments, the disease or disorder to be treated in accordance with the methods described herein is diabetes mellitus (including type I diabetes). Symptoms of diabetes mellitus include, but are not limited to, loss of weight, polyuria (frequent urination), polydipsia (increased thirst), polyphagia (increased hunger), cardiovascular disease, diabetic retinopathy, diabetic neuropathy, hyperosmolar nonketotic state, and diabetic ketoacidosis.

In some embodiments, the disease or disorder to be treated in accordance with the methods described herein is GVHD, such as acute GVHD, steroid-refractory acute GVHD (SR-aGVHD), treatment-refractory acute GVHD (TR-aGVHD), or chronic GVHD. See, e.g., Przepiorka et al., (2019) The Oncologist, 24:1-7 and Mothy et al., (2020) Blood, 136(17):1903-1906.

In some embodiments, an antibody or composition (e.g., pharmaceutical composition) of the present disclosure may be administered to a subject before, concurrently with, and/or after a transplantation. For example, as described in greater detail below, an antibody or composition of the present disclosure may be administered to increase the likelihood of a favorable treatment outcome, decrease the likelihood of an unfavorable outcome, and/or mitigate or prevent symptoms or unfavorable outcomes occurring before, concurrently with, or after the transplantation has been completed.

As used herein, treating an individual in need of a transplantation may refer to one or more of therapeutic treatment and prophylactic or preventative measures (e.g., increasing the likelihood of a favorable treatment outcome, such as graft survival, graft function, or decreasing the likelihood of an unfavorable outcome, such as an unfavorable response to treatment, or a condition that reduces the likelihood a favorable treatment, such as a transplantation, from occurring). Treating may include without limitation mitigating or preventing conditions and symptoms associated with a disorder or a condition, and/or problems or conditions that interfere with or limit an individual's access to treatment options of a disorder or a condition, such as sensitization, hypersensitization, high panel reactive antibodies (PRA) level and/or presence of pre-existing alloantibodies that limit availability of grafts to an individual awaiting a transplantation. Those in need of treatment include those already with the disorder or condition, as well as those in which the disorder or condition is to be prevented. Treatment of a disorder or condition may suppress immune-mediated events associated with the disorder or condition, ameliorate the symptoms of the disorder or condition, reduce the severity of the disorder or condition, alter the course of the disorder or condition progression, and/or ameliorate or cure the basic disorder or condition.

For example, successful treatment of an individual awaiting transplantation includes, but is not limited to, reducing the level of alloantibodies, reducing panel reactive antibodies (PRA), enabling the individual to have more cross-match compatible donors, increasing the likelihood or probability of the individual to receive a graft, shortening the expected waiting period of the individual for a graft, desensitizing the individual, lowering risk of transplant-associated symptoms or conditions (such as immune-mediated events as described below), or any combination thereof.

For example, successful treatment of an individual receiving a transplantation includes, but is not limited to, protection and maintenance of the transplanted organ or tissue for a long term, which comprises controlling, reversing, mitigating, delaying, or preventing one or more symptoms or undesirable conditions associated with the organ transplant, such as immune-mediated events, including, but not limited to, production of donor-specific alloantibodies (DSA), GVHD, antibody-mediated rejection (AMR), hyperacute graft rejection, chronic graft rejection, graft failure, and graft loss, as measured by functional or histological signs of the symptom or condition. A treatment capable of controlling a disorder or condition (e.g., graft rejection) may include a treatment that slows the progression of the disease process, when initiated after functional or histological signs of the disorder or condition (e.g., graft rejection) are observed. Further, a treatment capable of reversing a disease or condition (e.g., graft rejection) may include a treatment that, when initiated after functional or histological signs of the disease or condition (e.g., graft rejection) have appeared, reverses the disease process and returns functional and histological findings closer to normal. A treatment capable of “delaying progression” of a disorder or condition (e.g., graft rejection) may include deferring, hindering, slowing, retarding, stabilizing, and/or postponing development of the disorder or condition (e.g., graft rejection). This delay can be of varying lengths of time, depending on the history of the disease and/or subject being treated. As is evident to one skilled in the art, a sufficient or significant delay can, in effect, encompass prevention, in that the subject, e.g., a subject at risk for developing the disorder or condition, does not develop the disorder or condition.

In some embodiments, a transplantation of the present disclosure may be transplantation of one or more tissues or organs including without limitation bone marrow, kidney, heart, liver, neuronal tissue, lung, pancreas, skin, and intestine (e.g., small and/or large intestine, as well as any sub-tissues thereof).

In addition, antibodies of the disclosure are useful for preventing and/or treating certain disorders and diseases associated with or caused (in whole or in part) by increased proliferation and/or numbers of activated T-cells relative to the proliferation and/or numbers of activated T-cells found in healthy individuals or individuals not having the particular disorder or disease. Non-limiting examples of disorders and diseases that can be prevented and/or treated using the antibodies described herein include graft-versus-host disease and cases of transplantation rejection (including transplantation rejection using allogeneic or xenogeneic tissues) such as bone marrow transplantation, liver transplantation, kidney transplant, or the transplantation of any organ or tissue.

In some embodiments, an antibody or composition (e.g., pharmaceutical composition) of the present disclosure may be administered to a subject before, concurrently with, and/or after a transfusion. For example, as described in greater detail below, an antibody or composition of the present disclosure may be administered to increase the likelihood of a favorable treatment outcome, decrease the likelihood of an unfavorable outcome, and/or mitigate or prevent symptoms occurring before, concurrently with, or after the transfusion has been completed.

As used herein, treating an individual in need of a transfusion may refer to one or more of therapeutic treatment and prophylactic or preventative measures (e.g., increasing the likelihood of a favorable treatment outcome, such as replacement or supplementation of blood components/cells, or decreasing the likelihood of an unfavorable outcome, such as an unfavorable response to treatment, inefficacy of treatment, or immunological reaction, or a condition that reduces the likelihood a favorable treatment, such as a transfusion, from occurring). Treating may include without limitation mitigating or preventing conditions and symptoms associated with a disorder or a condition, and/or problems or conditions that interfere with or limit an individual's access to treatment options of a disorder or a condition. Those in need of treatment include those already with the disorder or condition, as well as those in which the disorder or condition is to be prevented. Treatment of a disorder or condition may suppress immune-mediated events associated with the disorder or condition, ameliorate the symptoms of the disorder or condition, reduce the severity of the disorder or condition, alter the course of the disorder or condition progression, and/or ameliorate or cure the disorder or condition.

In some embodiments, the transfusion is a transfusion comprising one or more of white blood cells, red blood cells, and platelets. In some embodiments, the transfusion comprises whole blood or one or more blood products, including without limitation white blood cells, red blood cells, platelets, fresh frozen plasma, cryoprecipitate or blood clotting factors, antibodies, and/or blood substitutes. Exemplary conditions that may be treated with a transfusion (e.g., transfusion of blood or a blood product) include without limitation hemorrhage or blood loss, reduced hematocrit or hemoglobin (e.g., anemia), sickle cell disease, thalassemia, blood supplementation during or after surgical procedures, cardiac disease, traumatic injury, deficiency of one or more blood factors (e.g., hemophilia, von Willebrand disease, hypofibrinogenemia, or a deficiency in factor II, V, VII, IX, X, or XI), conditions requiring fibrinogen supplementation (e.g., liver disease, blood transfusion, etc.), bone marrow failure, platelet function disorders, thrombocytopenia, immunodeficiency (e.g., from a therapy or disease), and the like. Descriptions of practices, dosing, responses, indications, and preparations related to transfusions may be found, e.g., in the American Red Cross Compendium of Transfusion Practice Guidelines.

In some embodiments, administration of an antibody of the disclosure, or a fragment or variant thereof, to a subject results in a reduction in one or more symptoms of a T-cell mediated inflammatory disease of the present disclosure in the subject.

In another embodiment, the disease or disorder to be treated in accordance with the methods described herein is a cancer. In some embodiments, the cancer is a T-cell cancer or neoplasm, such as a T-cell leukemia or lymphoma. In some embodiments, the cancer is an adult T-cell leukemia/lymphoma (ATLL) or cutaneous T-cell lymphoma (CTCL).

In some embodiments, prior to administration of the antibody of the disclosure, or the fragment or variant thereof, the subject has had one or more treatments, e.g., for a disease or disorder described herein, such as a T-cell mediated inflammatory disease, a condition associated with a transplantation or transfusion (e.g., transplant or transfusion rejection, GVHD, etc.), or a cancer. In some embodiments, prior to administration of the antibody of the disclosure, or the fragment or variant thereof, the subject has been treated with a corticosteroid.

### JAK Inhibitors

In some embodiments, an antibody of the present disclosure may be administered in combination with a Janus kinase (JAK) inhibitor.

The Janus kinase/signal transducer and activator of transcription (JAK/STAT) signaling pathway is known to be associated with the development and progression of many hematological and solid cancers (Waldmann and Chen (2017) Annu Rev Immunol 35:533-550; Vainchenker and Constantinescu (2013) Oncogene 32(21):2601-2613; Thomas et al., (2015) Br J Cancer, 113:365-71; O'Shea et al., (2013) N Engl J Med 368:161-70), and also to be integral to the effects of inflammatory cytokines on the immune system (Villarino et al., (2017) Nat Immunol, 18:374-384). STATs and their upstream activators, JAKs, are being extensively explored as targets for cancer and inflammatory disease therapy (Qureshy et al., (2020) J Cancer Metastasis Treat 6:27-44; Hosseini et al., (2020) J Cell Physiol. 235(9):5903-5924). Many JAK inhibitors have been or are actively being tested in clinical trials as monotherapy or in combination with other agents in patients with inflammatory diseases or cancers; at least five of these inhibitors are already Food and Drug Administration (FDA) approved for the treatment of inflammatory diseases or cancers including rheumatoid arthritis, ulcerative colitis, GvHD (Qureshy et al., (2020) J Cancer Metastasis Treat 6:27-44; Damsky et al., (2021) Journal of Allergy and Clinical Immunology 147(3):814-826), and myelofibrosis. Ruxolitinib is a JAK 1/2 inhibitor and is recently approved for the treatment of steroid-refractory acute GVHD (sr-aGvHD) and chronic GVHD (cGVHD).

In some embodiments, an antibody of the present disclosure may be administered to a subject in combination with a JAK inhibitor that inhibits JAK1, and/or JAK2, and/or JAK3. In some embodiments, the JAK inhibitor inhibits JAK1 and/or JAK2. In some embodiments, the JAK inhibitor inhibits JAK1 and/or JAK3. In some embodiments, the JAK inhibitor is a JAK1/JAK2 inhibitor, a JAK2/FLT3 inhibitor, a JAK2^{V617F} inhibitor, a JAK2 inhibitor, JAK1 inhibitor, or a JAK2/Src inhibitor, including pharmaceutically acceptable salts thereof. Exemplary and non-limiting descriptions of JAK inhibitors can be found in WO2007070514, WO2008157208, and WO2019171326, which are incorporated herein by reference in their entirety.

In some embodiments, the JAK inhibitor is ruxolitinib or a pharmaceutically acceptable salt thereof. As known in the art, ruxolitinib is the JAK1/JAK2 inhibitor (R)-3-(4-(7H-pyrrolo[2, 3-d]pyrimidin-4-yl)-1H-pyrazol-1-yl)-3-cyclopentylpropanenitrile, also named 3(R)-Cyclopentyl-3-[4-(7H-pyrrolo[2,3-d]pyrimidin-4-yl)-1H-pyrazol-1-yl]propanenitrile, of formula:

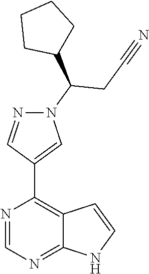 [SMILES: N#CC[C@H](C1CCCC1)n1cc(-c2ncnc3[nH]ccc23)cn1]

In some embodiments, ruxolitinib refers to the ruxolitinib phosphate salt. In some embodiments, ruxolitinib is in a unit dosage form (e.g., tablet). In some embodiments, ruxolitinib is administered orally.

In some embodiments, the JAK inhibitor is tofacitinib or a pharmaceutically acceptable salt thereof. Tofacitinib is an inhibitor of JAK1 and JAK3.

Administration of an antibody of the disclosure, or a fragment or variant thereof, in accordance with the methods described herein can be continuous or intermittent, depending, for example, upon the recipient's physiological condition, whether the purpose of the administration is therapeutic or prophylactic, and other factors known to skilled practitioners. The administration of an antibody of the disclosure, or a fragment or variant thereof, may be essentially continuous over a preselected period of time or may be in a series of spaced doses.

The dosage and frequency of administration of an antibody described herein, or a fragment or variant thereof, or a pharmaceutical composition thereof, is administered in accordance with the methods for preventing and/or treating as described herein while minimizing side effects. The exact dosage of an antibody described herein to be administered to a particular subject or a pharmaceutical composition thereof can be determined by a practitioner, in light of factors related to the subject that requires treatment. Factors which can be taken into account include the severity of the disease state, general health of the subject, age, and weight of the subject, diet, time and frequency of administration, combination(s) with other therapeutic agents or drugs, reaction sensitivities, and tolerance/response to therapy. The dosage and frequency of administration of an antibody described herein, or a fragment or variant thereof, or a pharmaceutical composition thereof, can be adjusted over time to provide sufficient levels of the antibody, or a fragment or variant thereof, or to maintain the desired effect.

The precise dose to be employed will also depend on the route of administration, and the seriousness of the disorder or disease, and should be decided according to the judgment of the practitioner and each patient's circumstances.

Effective doses can be extrapolated from dose-response curves derived from in vitro or animal model test systems.

In one embodiment, any of the antibodies of the disclosure, or a fragment or variant thereof, or pharmaceutical compositions thereof, may be formulated for administration by intraperitoneal, intravenous, subcutaneous, or intramuscular injections, or other forms of administration such as oral, mucosal, via inhalation, sublingually, etc. Parenteral administration, in one embodiment, is characterized by injection, either subcutaneously, intramuscularly or intravenously. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. In addition, if desired, the pharmaceutical compositions to be administered can also contain minor amounts of non-toxic auxiliary substances such as wetting or emulsifying agents, pH buffering agents, stabilizers, solubility enhancers, and other such agents. Other routes of administration may include, enteric administration, intracerebral administration, nasal administration, intraarterial administration, intracardiac administration, intraosseous infusion, intrathecal administration, intravenous infusion, subcutaneous implantation or injection, intramuscular administration, intrarectal administration, intravaginal administration, intragastrical administration, intratracheal administration, intrapulmonary administration and intraperitoneal administration. Preparations for parenteral administration include sterile solutions ready for injection, sterile dry soluble products, such as lyophilized powders, ready to be combined with a solvent just prior to use, including sterile suspensions ready for injection, sterile dry insoluble products ready to be combined with a vehicle just prior to use and sterile emulsions. The solutions can be either aqueous or nonaqueous. If administered intravenously, suitable carriers include physiological saline or phosphate buffered saline (PBS), water, and solutions containing thickening and solubilizing agents, such as glucose, polyethylene glycol, and polypropylene glycol and mixtures thereof.

In another embodiment, the present disclosure also contemplates administration of a composition comprising the antibodies of the present disclosure, or a fragment or variant thereof, conjugated to other molecules, such as detectable labels, or therapeutic or cytotoxic agents. The agents may include, but are not limited to radioisotopes, toxins, toxoids, inflammatory agents, enzymes, antisense molecules, peptides, cytokines, and chemotherapeutic agents. Methods of conjugating the antibodies with such molecules are generally known to those of skilled in the art. See, e.g., PCT publications WO 92/08495; WO 91/14438; WO 89/12624; U.S. Pat. No. 5,314,995; and EP 396,387.

In one embodiment, a composition of the disclosure comprises an antibody described herein, or a fragment or variant thereof, conjugated to a cytotoxic agent. Cytotoxic agents can include any agents that are detrimental to cells. An exemplary class of cytotoxic agents that can be conjugated to the antibodies, or fragments or variants thereof, disclosed herein may include, but are not limited to, paclitaxol, cytochalasin B, gramicidin D, ethidium bromide, emetine, mitomycin, etoposide, teniposide, vincristine, vinblastine, colchicine, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, and analogs or homologs thereof.

In some embodiments, the subject is a human.

### V. Pharmaceutical Compositions

The present disclosure also provides pharmaceutical compositions comprising antibodies described herein, or fragments or variants thereof, and/or a JAK inhibitor, along with pharmaceutically acceptable carriers or excipients. The pharmaceutical compositions may find use, e.g., in the methods, uses, and/or kits of the present disclosure.

Pharmaceutically acceptable carriers or excipients are known in the art, and are relatively inert substances that facilitate administration of a pharmacologically effective substance. For example, an excipient can give form or consistency, or act as a diluent. Suitable excipients include but are not limited to stabilizing agents, wetting and emulsifying agents, salts for varying osmolarity, encapsulating agents, buffers, and skin penetration enhancers. In certain embodiments, an antibody described herein, or fragments or variants thereof, is in a liquid pharmaceutical composition. Liquid pharmaceutically administrable compositions can, for example, be prepared by dissolving, dispersing, or otherwise mixing an antibody described herein in a carrier, such as, for example, water, saline, aqueous dextrose, glycerol, glycols, ethanol, and the like, to thereby form a solution or suspension. If desired, the pharmaceutical composition to be administered can also contain minor amounts of nontoxic auxiliary substances such as wetting agents, emulsifying agents, solubilizing agents, and pH buffering agents, and the like. Excipients as well as formulations for parenteral and nonparenteral drug delivery are set forth in Remington, The Science and Practice of Pharmacy 20th Ed. Mack Publishing (2000).

The pharmaceutical compositions are provided for administration to human and animal subjects in unit dosage forms, such as sterile parenteral solutions or suspensions containing suitable quantities of an antibody described herein, or fragments or variants thereof. The antibody (or fragments or variants thereof) and/or JAK inhibitor is, in one embodiment, formulated and administered in unit-dosage forms or multiple-dosage forms. Unit-dose forms as used herein refers to physically discrete units suitable for human and animal subjects and packaged individually as is known in the art. Each unit-dose contains a predetermined quantity of the antibody (or fragments or variants thereof) and/or JAK inhibitor sufficient to produce the desired therapeutic effect, in association with the required pharmaceutical carrier, vehicle or diluent. Examples of unit-dose forms include ampoules and syringes. Unit-dose forms can be administered in fractions or multiples thereof. A multiple-dose form is a plurality of unit-dosage forms packaged in a single container to be administered in segregated unit-dose forms. Examples of multiple-dose forms include vials, or bottles of pints or gallons. Hence, a multiple dose form is a multiple of unit-doses which are not segregated in packaging.

The concentration of an antibody of the disclosure (or fragments or variants thereof) and/or a JAK inhibitor, in the pharmaceutical compositions will depend on, e.g., the physicochemical characteristics of the antibody (or fragments or variants thereof) and/or the JAK inhibitor, the dosage schedule, and amount administered as well as other factors known to those of skill in the art. In some embodiments, the pharmaceutical compositions provide a dosage of from about 0.001 mg to about 100 mg of antibody (or fragments or variants thereof) per kilogram of body weight per day. Pharmaceutical dosage unit forms can be prepared to provide from about 0.001 mg to about 100 mg, and/or a combination of other optional essential ingredients per dosage unit form.

In some embodiments, the present disclosure provides antibodies (or fragments or variants thereof) and/or JAK inhibitors, and compositions thereof (such as the pharmaceutical compositions described herein) for use in any of the methods described herein, whether in the context of use as a medicament and/or use for manufacture of a medicament.

### VI. Kits

Certain aspects of the present disclosure are related to kits or articles of manufacture that comprise an antibody of the present disclosure (or fragments or variants thereof) and/or a JAK inhibitor. Optionally, the kits described herein may contain one or more pharmaceutically acceptable carriers, such as the exemplary carriers described herein. In some embodiments, a kit of the present disclosure includes a pharmaceutical composition of the present disclosure. Kits described herein may find use, e.g., in the methods or uses of the present disclosure.

Kits may optionally provide additional components such as buffers and interpretive information. Normally, the kit comprises a container and a label or package insert(s) on or associated with the container. The containers may be unit doses, bulk packages (e.g., multi-dose packages) or sub-unit doses. Instructions supplied in the kits of the present disclosure are typically written instructions on a label or package insert (e.g., a paper sheet included in the kit), but machine-readable instructions (e.g., instructions carried on a magnetic or optical storage disk) are also acceptable.

In some embodiments, the kits include a package insert comprising instructions for administration of the antibody, or fragments or variants thereof, to treat a disease, disorder or condition described herein. In some embodiments, the kits include a package insert comprising instructions for administration of the antibody, or fragments or variants thereof, to treat a T-cell mediated inflammatory disease. In some embodiments, the kits include a package insert comprising instructions for administration of the antibody, or fragments or variants thereof, before, concurrently with, and/or after a transfusion or transplantation. In some embodiments, the kits include a package insert comprising instructions for administration of the antibody, or fragments or variants thereof, to treat a cancer, e.g., as described herein. In some embodiments, the kits further include instructions (e.g., in a package insert) for administration of the antibody, or fragments or variants thereof, in combination with a JAK inhibitor. In some embodiments, the kits include an antibody of the present disclosure (or fragments or variants thereof). In some embodiments, the kits include a JAK inhibitor. In some embodiments, the kits include an antibody of the present disclosure (or fragments or variants thereof) and a JAK inhibitor.

The kits of the present disclosure are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer), or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is an antibody described herein, or a fragment or variant thereof. The container may further comprise a second pharmaceutically active agent, e.g., a JAK inhibitor. In some embodiments, a kit may further include any other material or device useful in a treatment (e.g., a transfusion or transplantation), including without limitation one or more containers, tubing, sterilizing agents or equipment, cannulae, syringes, and the like.

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1: Anti-hPSGL-1 Bivalent mAb 15A7H Functional Activity Measured with Annexin V+PI Staining for Apoptotic Cells

### Materials and Methods

### Human T-Cell Preparation

Human peripheral blood mononuclear cells (PBMCs) were freshly isolated from whole blood by Ficoll-Hypaque gradient centrifugation (Ficoll® PAQUE PLUS solution, GE Healthcare, Cat #17-1440-03) at 2400 rpm for 15 min at room temperature. The buffy coat layer containing mononuclear cells was collected and washed 3 times with PBS to minimize platelet contamination. The harvested PBMCs were used for the generation of activated T cells by incubating with phytohemagglutinin (PHA; 3 μg/ml, SIGMA, Cat. #11249738001) for 2 days and followed by maintaining in medium containing recombinant human Interleukin-2 (5 ng/mL, R&D System, Cat. #202-IL-050) for additional 4-6 days for apoptosis assays.

In vitro Functional Activity Measured with Annexin V+PI Staining for Apoptotic Cells in SP2/O-hPSGL-1 and Activated Primary T Cells

For study of hPSGL-1 transfectant cells, Sp2/0-hPSGL-1 or the parental Sp2/0 cells (1×10⁵) were seeded into 96-well plates. For the study of human primary T cells, each test well was seeded with 1×10′ activated T cells (Day 7 post PHA stimulation) in a 96-well plate. Anti-PSGL-1 bivalent mAb 15A7H (an IgG4 isotype antibody) is described in International Application Pub. No. WO 2012/174001. Aliquots of 15A7H antibody were generated at final concentrations ranging from 30 (Sp2/0) or 10 (activated T) to 0.04 g/mL. Isotype control antibody was added only with the highest concentration of 10 or 30 g/mL. A cross-linker (CL) antibody, a mouse anti-human IgG Fcγ fragment specific (Jackson ImmunoResearch Cat. No. 209-005-098), at half of the 15A7H or isotype control antibody concentration, was freshly prepared in complete RPMI (10% FBS, 1% Penicillin/Streptomycin and 2 ng/mL IL-2) and added to the test wells at a final volume of 100 L. An additional set of wells were added with complete medium at a final volume of 100 L for no CL treatment comparison. The sample with no antibody (untreated), CL only and positive control were also included for testing. The plate of hPSGL1 transfectant cell study was incubated at 37° C. for 5 hr and activated T cell for 24 hr.

For the apoptotic cell measurement, an Annexin-V−FITC Apoptosis Detection Kit (Strong Biotech, Cat. No. AVK250) was used and followed by manufacturer's instructions. In brief, the treated cells were harvested and resuspended in 100 μl of Annexin V binding buffer containing 0.5 μl of Annexin V−FITC at room temperature. After 15 min incubation in the dark, the cells were washed twice with 200 μl of Annexin V binding buffer. One microliter of propidium iodide (PI) per sample was added before FACS analysis. All flow cytometric analyses were performed by a BD-LSR flow cytometer (Becton Dickinson) using Cell Quest software. The Annexin V positive and/or PI positive cells were considered apoptotic cells.

### Results

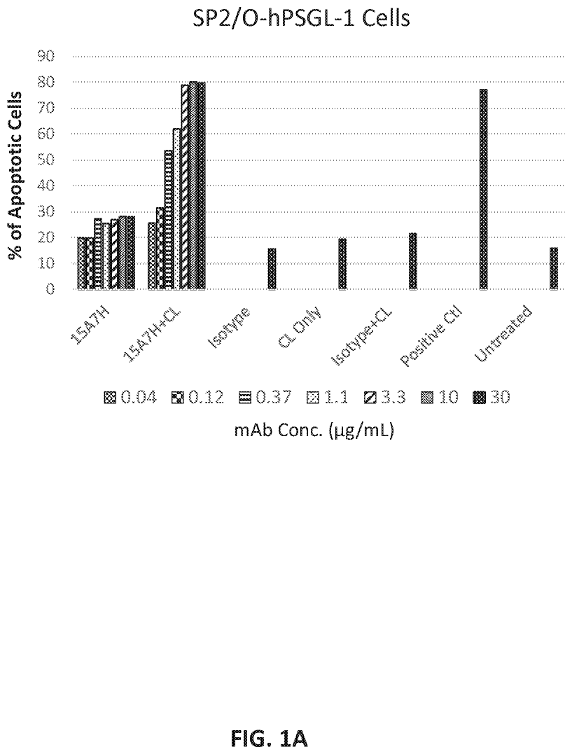

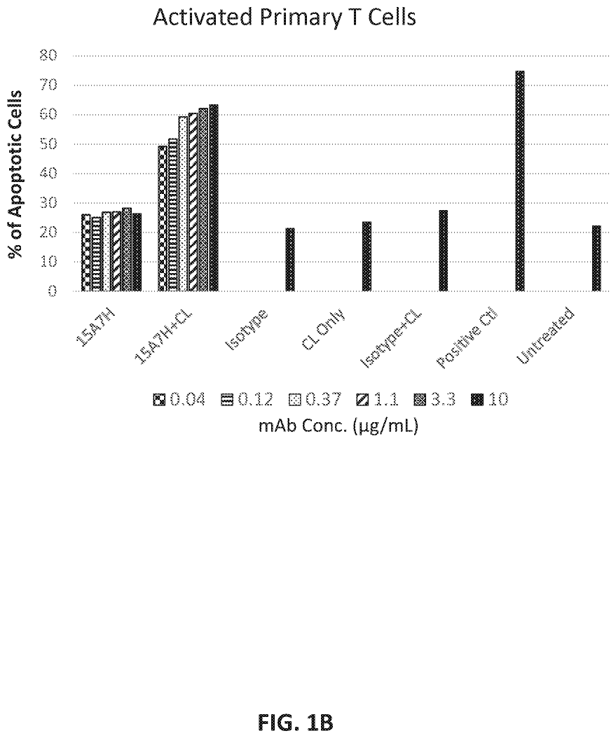

Certain agonistic anti-PSGL-1 antibodies have been developed (see, e.g., U.S. Pat. No. 7,604,800; WO2012/174001; Chen et al., Cross-Linking of P-Selectin Glycoprotein Ligand-1 Induces Death of Activated T Cells, Blood 104(10):3233-3242; and Huang et al., A Novel Apoptosis-Inducing Anti-PSGL-1 Antibody for T Cell-Mediated Diseases, Eur. J. Immunol. 2005, 35(7):2239-49). As shown in FIGS. 1A-1B, cross-linking was required for anti-PSGL-1 bivalent mAb 15A7H to achieve better apoptosis-inducing effect in transfectant Sp2/0-hPSGL1 and activated primary T cells. Annexin V and PI staining were used to measure apoptotic and dead cells, respectively, using flow cytometry. Results with untreated cells are shown in the rightmost panel of the figures, and results using anti-human PSGL-1 mAb in the absence of cross-linker are shown in the leftmost panel of the figures. Little to almost no cytotoxicity in the absence of a cross-linker was observed. Results using anti-PSGL-1 bivalent mAb 15A7H in the presence of cross-linker are shown in the second panel from the left in the figures. A significant dose-dependent change in the percentage of apoptotic cells was observed. Without wishing to be bound to theory, it was hypothesized that a pentameric mAb with multivalency, such as IgM, could possibly result a better potency than a bivalent mAb.

### Example 2: Anti-PSGL-1 IgM Antibody Generation

Plasmid constructs that can express pentameric IgM binding molecules that can specifically bind to hPSGL-1 were produced by the following method.

DNA constructs were synthesized at ATUM Bio using a proprietary process. DNA fragments encoding the VL and VH regions of 15A7H (SEQ ID NOs 15 and 16, respectively) were synthesized with a restriction site introduced on the ‘5 end and on the 3’ end for subcloning into heavy chain and light chain expression vectors. The gene synthesis process assembled oligonucleotides using template free PCR. The amplicon was purified and cloned using standard cloning methods. Ligated DNA was transformed into E. coli, and corresponding DNA was purified for quality control through Sanger DNA sequencing. The plasmid constructs encoding the IgM heavy chains, light chains, and J-chain were cotransfected into HEK293.sus cells, and cells that express the 15A7H-IgM antibody with J-chain, were selected, all according to standard methods.

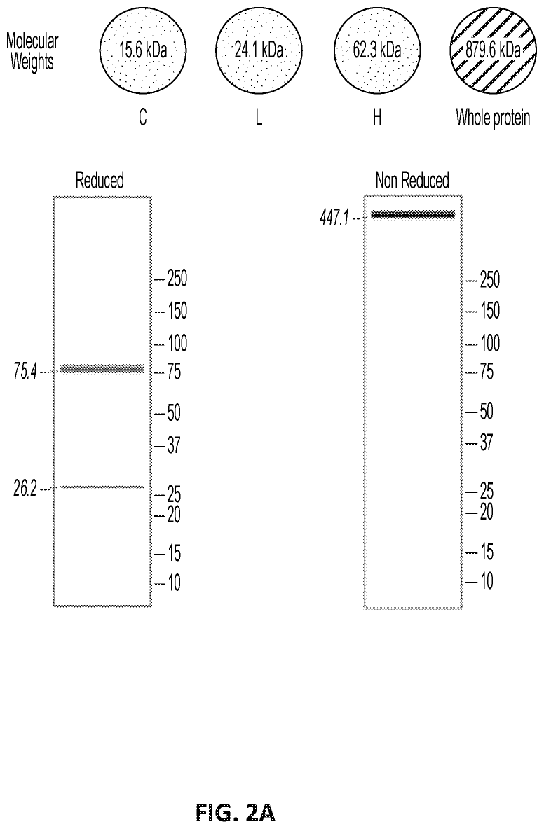

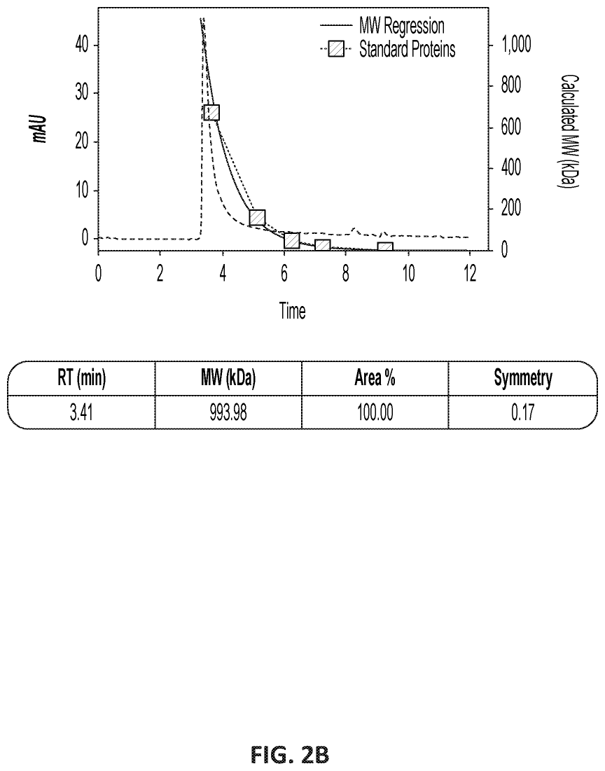

CaptureSelect IgM resin was used to capture the expressed IgM proteins and size exclusion chromatography (SEC) was used to remove the incompletely formed IgM species. The purity and approximate molecular mass of the purified 15A7H-IgM were assessed with SDS-PAGE under reducing and non-reducing condition and HPLC-SEC analysis (FIGS. 2A-2B). These results demonstrated the purification of 15A7H-IgM.

### Example 3: Binding Activity of 15A7H-IgM

### Materials and Methods

96-well NUNC-Immuno plates (Thermo Fisher, Cat #442404) were coated with 100 μL per well of 0.5, 0.05, or 0.03 pg/mL human PSGL-1 (extracellular domain) recombinant protein (SinoBiological, 13863-H08H) overnight at 4° C. Plates were then washed with Wash Buffer (1×PBS with 0.05% Tween 20, pH 7.4) and blocked with 0.5% (w/v) BSA-PBS for 60 min at room temperature on a plate shaker set to 300 rpm. After blocking, 100 μL of serial dilutions of 15A7H-IgM, 15A7H-IgG4, and controls at concentration range of 5000 pM-4.89 pM were added to the wells and incubated at room temperature for 1 hour. The bound IgM or IgG4 was revealed by adding a pre-mix of goat anti-human Ig kappa light chain specific antibody (Lifespan Bioscience, Cat #LS-C59091) and Peroxidase-conjugated antibody specific to Goat IgG (Jackson ImmunoResearch, Cat #305-035-003) in assay diluent buffer of PBS/0.1% (w/v) BSA/0.05% (w/v) Tween 20) for 60 min incubation at room temperature. After 5 final washes using Wash Buffer, the plates were read out using TMB substrate (followed the instruction manual of BD OptEIA. Cat. 555214) by incubating the wells with 100 L each of freshly prepared TMB Working Solution for 5 min followed by the addition of sulfuric acid to stop the reaction. Finally, the data of absorbance at 450 nm (OD450 nm) were collected on an ELISA plate reader and analyzed with SoftMax using a 4-parameter logistic model.

### Results

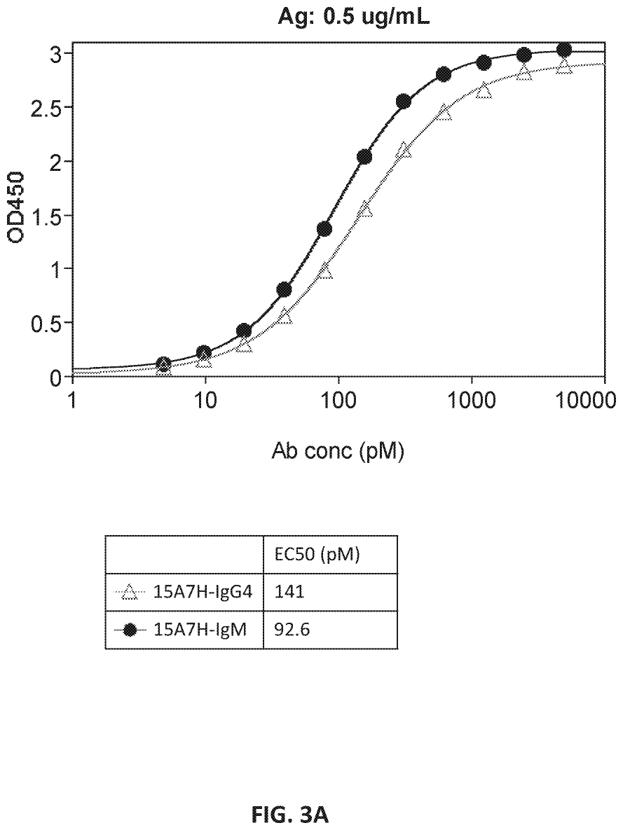

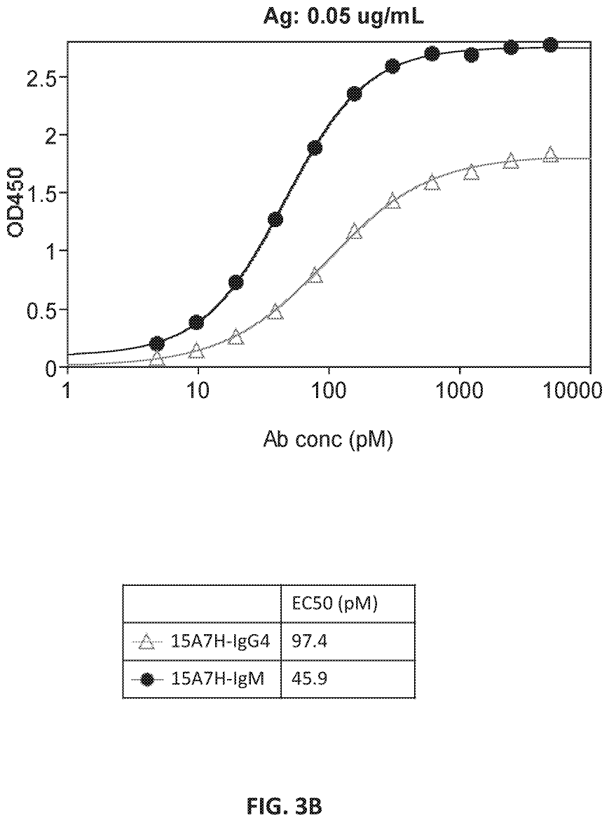

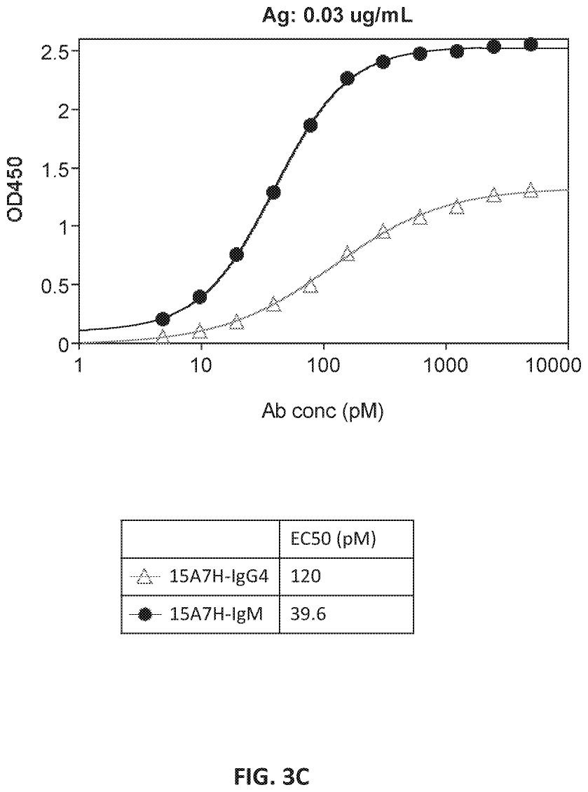

The results are shown in the FIGS. 3A-3C, comparing IgM vs. IgG4 by molar concentrations. The 15A7H-IgM antibody exhibited more effective binding at 0.5-0.03 pg/mL antigen densities, especially at the lower PSGL-1 antigen concentrations.

### Example 4: In Vitro Functional Activity Measured with Annexin V+PI Staining for Apoptotic Cells Detection by Flow Cytometry

### Materials and Methods

Three healthy donors PBMCs were isolated from whole blood by Ficoll-Hypaque gradient centrifugation (Ficoll® PAQUE PLUS solution, GE Healthcare, Cat #17-1440-03) at 2,200 rpm for 15 min at room temperature. The buffy coat layer containing mononuclear cells was collected and washed 3 times with PBS to minimize platelet contamination. The harvested PBMCs were used for the generation of activated T cells by incubating with Phytohaemagglutinin (PHA) (3 pg/mL, SIGMA, Cat. #11249738001) for 2 days and followed by maintaining in RPMI complete medium containing with 10% FBS, 2-ME and 5 ng/mL of recombinant human Interleukin-2 (IL-2, R&D System, Cat. No. 202-IL-050) until the assay was conducted on the indicated date.

Each test well was seeded with 1×10′ activated T cells (Day 5-7) in a 96-well plate. Aliquots of IgG4 or IgM antibody at final concentrations ranging from 100 to 0.00128 nM. Isotype control antibody was added only with the highest concentration of 100 nM. A cross-linker (CL, a mouse anti-human IgG antibody), at half of the IgG4 or control antibody concentration, was freshly prepared in complete RPMI (10% FBS, 1% Penicillin/Streptomycin and 2 ng/mL IL-2) and added to the test wells at a final volume of 100 L. The plate was incubated at 37° C. for 24 hr.

Annexin-V and Propidium Iodide (PI) staining procedures were carried out using Annexin-V FITC Apoptosis Detection Kit (Strong Biotech, AVK250), according to the manufacturer's instruction. The Annexin-V FITC and PI associated with the antibody-treated cells was read in a Flow cytometer, and the resultant Annexin V or PI staining data were acquired by the BD FACSuite and analyzed by the FlowJo_v10.9 software. The Annexin V positive and/or PI positive cells were considered apoptotic cells.

### Results

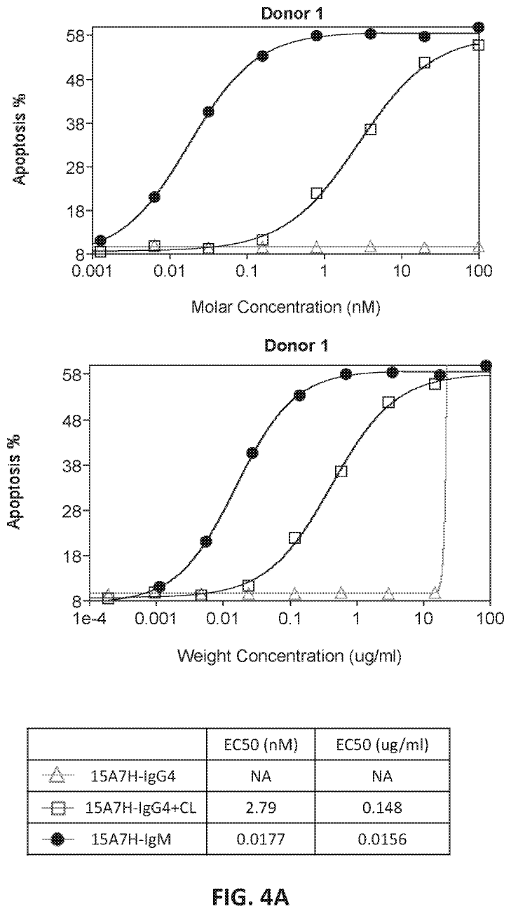

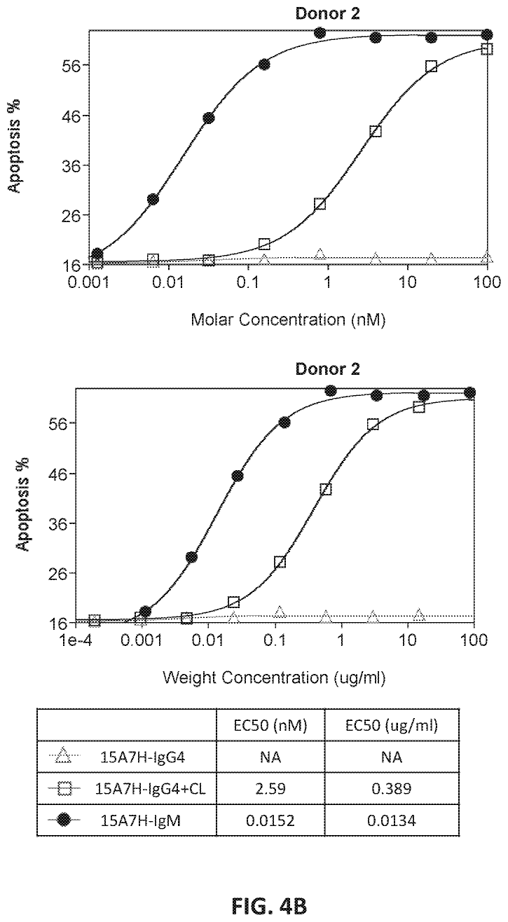

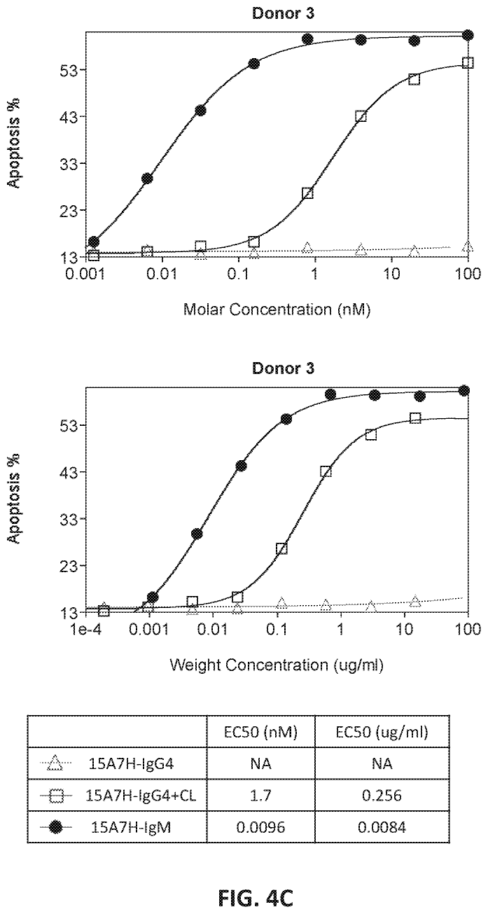

The percentage of apoptosis increase over isotype control treatment were plotted as % apoptosis versus concentration of antibody in FIGS. 4A-4C. These results show the superior in vitro activity of multimeric anti-PSGL-1 antibodies.

As shown in FIGS. 4A-4C, very minimum increase of apoptotic cells was detected in the cells treated with 15A7H-IgG4 antibody alone. Only when the mouse anti-human IgG was included as a cross-linker (CL) in the treatment, 15A7H-IgG4 antibody can induce cell apoptosis more effectively as significant increase in percentage of Annexin V+PI Staining positive cells was resulted (15A7H+CL in FIGS. 4A-4C), and this increase of apoptotic cells was dose-dependent. However, when pentameric 15A7H-IgM was used, the dose-dependent cell apoptosis effect was even much more efficient, as the apoptosis responsive curve resulted was much shifted toward to lower concentration range than 15A7H combining cross-linker in the comparison figures made either with molar concentration- or weight concentration-based.

The calculated EC50 from the three treatment responsive curves of each donor and the fold difference of IgG4+CL over IgM were summarized in TABLE A.

TABLE A
Calculated EC50 values for 15A7H-IgG4 + CL vs. 15A7H-IgM.

| Fold Difference | EC50 (nM) | | | EC50 (μg/mL) | | |
|---|---|---|---|---|---|---|
| of EC50 Based | | | Fold | | | Fold |
| on Molar/ | 15A7H- | 15A7H- | Difference of | 15A7H- | 15A7H- | Difference of |
| Weight Conc | IgG4 + CL | IgM | IgG4 + CL/IgM | IgG4 + CL | IgM | IgG4 + CL/IgM |
| Donor 1 | 2.79 | 0.0177 | 158 | 0.418 | 0.0156 | 27 |
| Donor 2 | 2.59 | 0.0152 | 170 | 0.389 | 0.0134 | 29 |
| Donor 3 | 1.7 | 0.0096 | 177 | 0.256 | 0.00844 | 30 |

As shown in Table A, when molar concentration of EC50 was used for potency comparison, there was at least a 150-fold difference observed between IgM and IgG4+CL treatment. Even when weight concentration of EC50 was used for potency comparison, there was still an approximately 30-fold difference observed between IgM and IgG4+CL treatment. This indicates that 15A7H-IgM antibody can induce apoptosis in human activated T cell at a higher potency than an equivalent amount (based on weight concentration) of a bivalent IgG4 antibody.

Although the foregoing embodiments have been described in some detail by way of illustration and example for purposes of clarity of understanding, the descriptions and examples should not be construed as limiting the scope of the present disclosure.

What is claimed is:

1. An IgM antibody that binds to a human P-selectin glycoprotein ligand-1 (PSGL-1) protein, wherein the antibody comprises a heavy chain comprising a heavy chain variable domain and a light chain comprising a light chain variable domain, wherein:
the light chain variable domain comprises a CDR-L1 comprising the amino acid sequence RSSQSIVHNDGNTYFE (SEQ ID NO: 1), a CDR-L2 comprising the amino acid sequence KVSNRFS (SEQ ID NO: 2), and a CDR-L3 comprising the amino acid sequence FQGSYVPLT (SEQ ID NO: 3); and
the heavy chain variable domain comprises a CDR-H1 comprising the amino acid sequence SFGMH (SEQ ID NO: 4), a CDR-H2 comprising the amino acid sequence YINGGSSTIFYANAVKG (SEQ ID NO: 5), and a CDR-H3 comprising the amino acid sequence YASYGGGAMDY (SEQ ID NO: 6).

2. The antibody of claim 1, wherein the antibody induces apoptosis of activated T cells to a greater extent than a control anti-PSGL-1 antibody or antibody fragment.

3. The antibody of claim 2, wherein the control anti-PSGL-1 antibody or antibody fragment is a bivalent anti-PSGL-1 antibody or antibody fragment.

4. The antibody of claim 2 or claim 3, wherein the control anti-PSGL-1 antibody comprises a heavy chain variable domain and a light chain variable domain, wherein the light chain variable domain comprises a CDR-L1 comprising the amino acid sequence RSSQSIVHNDGNTYFE (SEQ ID NO: 1), a CDR-L2 comprising the amino acid sequence KVSNRFS (SEQ ID NO: 2), and a CDR-L3 comprising the amino acid sequence FQGSYVPLT (SEQ ID NO: 3); and the heavy chain variable domain comprises a CDR-H1 comprising the amino acid sequence SFGMH (SEQ ID NO: 4), a CDR-H2 comprising the amino acid sequence YINGGSSTIFYANAVKG (SEQ ID NO: 5), and a CDR-H3 comprising the amino acid sequence YASYGGGAMDY (SEQ ID NO: 6).

5. The antibody of claim 4, wherein the control anti-PSGL-1 antibody comprises a heavy chain variable domain comprising the amino acid sequence of SEQ ID NO: 16 and a light chain variable domain comprising the amino acid sequence of SEQ ID NO: 15.

6. The antibody of claim 4 or claim 5, wherein the control anti-PSGL-1 antibody comprises a heavy chain comprising the amino acid sequence of SEQ ID NO: 24 and a light chain comprising the amino acid sequence of SEQ ID NO: 23.

7. The antibody of any one of claims 1-6, wherein the human PSGL-1 protein comprises the amino acid sequence of SEQ ID NO: 21 or SEQ ID NO: 22.

8. The antibody of any one of claims 1-7, wherein the antibody is a chimeric, humanized or human antibody.

9. The antibody of any one of claims 1-8, wherein the antibody is a bispecific or multi-specific antibody.

10. The antibody of any one of claims 1-9, wherein the heavy chain variable domain comprises the amino acid sequence of SEQ ID NO: 16, or an amino acid sequence having at least about any of 80%, 85%, 90%, 95%, or 99% identity thereto.

11. The antibody of any one of claims 1-10, wherein the light chain variable domain comprises the amino acid sequence of SEQ ID NO: 15, or an amino acid sequence having at least about any of 80%, 85%, 90%, 95%, or 99% identity thereto.

12. The antibody of any one of claims 1-11, wherein the antibody is a multivalent antibody.

13. The antibody of any one of claims 1-12, wherein the antibody is a pentameric IgM antibody.

14. The antibody of any one of claims 1-12, wherein the antibody is a hexameric IgM antibody.

15. The antibody of any one of claims 1-14, wherein the antibody comprises a J-chain, or a fragment or variant thereof.

16. The antibody of claim 15, wherein the J-chain comprises the amino acid sequence of SEQ ID NO: 19, or an amino acid sequence having at least about any of 80%, 85%, 90%, 95%, or 99% identity thereto.

17. The antibody of any one of claims 1-16, wherein the heavy chain comprises an IgM heavy chain constant region, or a fragment or variant thereof, optionally wherein the IgM heavy chain constant region is a human IgM heavy chain constant region or a fragment or variant thereof.

18. The antibody of claim 17, wherein the IgM heavy chain constant region comprises a Cμ1 domain, a Cμ2 domain, a Cμ3 domain, and/or a Cμ4 domain.

19. The antibody of any one of claims 1-18, wherein the antibody has reduced complement-dependent cytotoxicity (CDC) activity as compared to a corresponding wild-type IgM antibody.

20. The antibody of claim 19, wherein the antibody comprises one or more amino acid substitutions in the IgM constant region that confer reduced CDC activity as compared to the corresponding wild-type IgM antibody.

21. The antibody of any one of claims 18-20, wherein the IgM heavy chain constant region comprises a tailpiece polypeptide C-terminal to the Cμ4 domain.

22. The antibody of claim 21, wherein the IgM heavy chain constant region comprises the Cμ3 domain, the Cμ4 domain, and the tailpiece polypeptide C-terminal to the Cμ4 domain.

23. The antibody of claim 22, wherein the antibody further comprises the Cμ1 domain and/or the Cμ2 domain.

24. The antibody of any one of claims 1-23, wherein the heavy chain comprises an IgM heavy chain constant region comprising the amino acid sequence of SEQ ID NO: 18, or an amino acid sequence with at least about any of 80%, 85%, 90%, 95%, or 99% identity thereto.

25. The antibody of any one of claims 1-24, wherein the light chain comprises a human kappa light chain constant region.

26. The antibody of any one of claims 1-25, wherein the light chain comprises the amino acid sequence of SEQ ID NO: 17, or an amino acid sequence with at least about any of 80%, 85%, 90%, 95%, or 99% identity thereto.

27. The antibody of any one of claims 1-26, wherein the heavy chain comprises the amino acid sequence of SEQ ID NO: 20, or an amino acid sequence with at least about any of 80%, 85%, 90%, 95%, or 99% identity thereto.

28. One or more isolated nucleic acids encoding the antibody of any one of claims 1-27.

29. A vector comprising the one or more nucleic acids of claim 28.

30. A host cell comprising the one or more nucleic acids of claim 28, or the vector of claim 29.

31. A method of producing an anti-PSGL-1 antibody, comprising culturing the host cell of claim so that the antibody is produced.

32. The method of claim 31, further comprising recovering the antibody from the host cell.

33. An anti-PSGL-1 antibody produced by the method of claim 31 or claim 32.

34. A pharmaceutical composition comprising the antibody of any one of claims 1-27 and a pharmaceutically acceptable carrier.

35. A kit comprising the antibody of any one of claims 1-27 and an optional pharmaceutically acceptable carrier.

36. The kit of claim 35, further comprising a package insert comprising instructions for administration of the antibody to treat a T-cell mediated inflammatory disease in a subject in need thereof.

37. The kit of claim 35, further comprising a package insert comprising instructions for administration of the antibody before, concurrently with, and/or after a transfusion or transplantation in a subject in need thereof.

38. The kit of claim 35, further comprising a package insert comprising instructions for administration of the antibody to treat a cancer in a subject in need thereof.

39. The kit of any one of claims 36-38, wherein the package insert further comprises instructions for using the antibody in combination with a Janus kinase (JAK) inhibitor.

40. The kit of any one of claims 35-39, wherein the kit further comprises a JAK inhibitor.

41. A method of treating a T-cell mediated inflammatory disease, the method comprising administering to a subject in need thereof a therapeutically effective amount of the antibody of any one of claims 1-27.

42. A method of treating a subject in need of a transfusion or transplantation, comprising administering to a subject in need thereof a therapeutically effective amount of the antibody of any one of claims 1-27 before, concurrently with, and/or after a transfusion or transplantation.

43. A method of treating a cancer, the method comprising administering to a subject in need thereof a therapeutically effective amount of the antibody of any one of claims 1-27.

44. The method of any one of claims 41-43, further comprising administering a JAK inhibitor to the subject.

45. The antibody of any one of claims 1-27 for use in treating a T-cell mediated inflammatory disease in a subject in need thereof.

46. The antibody of any one of claims 1-27 for use in treating a subject in need of a transfusion or transplantation.

47. The antibody of any one of claims 1-27 for use in treating a cancer in a subject in need thereof.

48. The antibody for use of any one of claims 45-47, wherein the antibody is for use in combination with a JAK inhibitor.

49. Use of the antibody of any one of claims 1-27 in the manufacture of a medicament for treating a T-cell mediated inflammatory disease in a subject in need thereof.

50. Use of the antibody of any one of claims 1-27 in the manufacture of a medicament for treating a subject in need of a transfusion or transplantation.

51. Use of the antibody of any one of claims 1-27 in the manufacture of a medicament for treating a cancer in a subject in need thereof.

52. The use of any one of claims 49-51, wherein the antibody is to be administered in combination with a JAK inhibitor.

53. The kit of claim 36, the method of claim 41, the antibody for use of claim 45, or the use of claim 49, wherein the T-cell mediated inflammatory disease is an autoimmune disease.

54. The kit any one of claims 36, 39, and 40, the method of claim 41 or claim 44, the antibody for use of claim 45 or claim 48, or the use of claim 49 or claim 52, wherein the T-cell mediated inflammatory disease is selected from the group consisting of: psoriasis, psoriatic arthritis, rheumatoid arthritis, Crohn's disease, ankylosing spondylitis, type I diabetes, ulcerative colitis, multiple sclerosis, allergy, atopic dermatitis, asthma, graft versus host disease (GVHD), vitiligo, alopecia areata, Steven Johnson Syndrome (SJS), Drug reaction with eosinophilia and systemic symptoms (Dress), T-cell mediated skin diseases, juvenile arthritis, lupus, inflammatory bowel disease, myasthenia gravis, immunoglobulin nephropathies, myocarditis, and autoimmune thyroid disorder.

55. The kit, method, antibody, or use of claim 54, wherein the GVHD is acute GVHD, steroid-refractory acute GVHD (SR-aGVHD), treatment-refractory acute GVHD (TR-aGVHD), or chronic GVHD.

56. The kit any one of claims 37, 39, and 40, the method of claim 42 or claim 44, the antibody for use of claim 46 or claim 48, or the use of claim 50 or claim 52, wherein the transplantation is a transplantation of a tissue selected from the group consisting of bone marrow, kidney, heart, liver, neuronal tissue, lung, pancreas, skin, and intestine.

57. The kit any one of claims 37, 39, and 40, the method of claim 42 or claim 44, the antibody for use of claim 46 or claim 48, or the use of claim 50 or claim 52, wherein the transfusion is a transfusion comprising one or more of white blood cells, red blood cells, and platelets.

58. The kit of any one of claims 38-40, the method of claim 43 or claim 44, the antibody for use of claim 47 or claim 48, or the use of claim 51 or claim 52, wherein the cancer is a T-cell neoplasm, optionally a T-cell leukemia or T-cell lymphoma.

59. The kit, method, antibody, or use of claim 58, wherein the T-cell leukemia or lymphoma is adult T-cell leukemia/lymphoma (ATLL) or cutaneous T-cell lymphoma (CTCL).

60. The kit of any one of claims 35-40 and 53-59, the method of any one of claims 41-44 and 53-59, the antibody for use of any one of claims 45-48 and 53-59, or the use of any one of claims 49-59, wherein the subject is a human.

61. The kit of any one of claims 39-40 and 53-60, the method of any one of claims 44 and 53-60, the antibody for use of any one of claims 48 and 53-60, or the use of any one of claims 52-60, wherein the JAK inhibitor inhibits JAK1 and/or JAK2.

62. The kit of any one of claims 39-40 and 53-60, the method of any one of claims 44 and 53-60, the antibody for use of any one of claims 48 and 53-60, or the use of any one of claims 52-60, wherein the JAK inhibitor inhibits JAK1 and/or JAK3.

63. The kit of any one of claims 39-40 and 53-60, the method of any one of claims 44 and 53-60, the antibody for use of any one of claims 48 and 53-60, or the use of any one of claims 52-60, wherein the JAK inhibitor is ruxolitinib.

64. The kit of any one of claims 39-40 and 53-60, the method of any one of claims 44 and 53-60, the antibody for use of any one of claims 48 and 53-60, or the use of any one of claims 52-60, wherein the JAK inhibitor is tofacitinib.


SEQUENCE LISTING (24 sequences)

SEQ ID NO: 1 - protein, 16 aa, synthetic construct
RSSQSIVHNDGNTYFE

SEQ ID NO: 2 - protein, 7 aa, synthetic construct
KVSNRFS

SEQ ID NO: 3 - protein, 9 aa, synthetic construct
FQGSYVPLT

SEQ ID NO: 4 - protein, 5 aa, synthetic construct
SFGMH

SEQ ID NO: 5 - protein, 17 aa, synthetic construct
YINGGSSTIFYANAVKG

SEQ ID NO: 6 - protein, 11 aa, synthetic construct
YASYGGGAMDY

SEQ ID NO: 7 - protein, 23 aa, synthetic construct
DIQMTQSPSSLSASVGDRVTITC

SEQ ID NO: 8 - protein, 15 aa, synthetic construct
WYQQKPGKAPKLLIY

SEQ ID NO: 9 - protein, 32 aa, synthetic construct
GVPSRFSGSGSGTHFTLTISSLQPEDFATYYC

SEQ ID NO: 10 - protein, 10 aa, synthetic construct
FGQGTKVEIK

SEQ ID NO: 11 - protein, 30 aa, synthetic construct
EVQLVESGGGLVQPGGSLRLSCAASGFTFS

SEQ ID NO: 12 - protein, 14 aa, synthetic construct
WVRQAPGKGLEWVA

SEQ ID NO: 13 - protein, 32 aa, synthetic construct
RFTISRDNAKNTLYLQMNSLRAEDTAVYYCAR

SEQ ID NO: 14 - protein, 11 aa, synthetic construct
WGQGTLVTVSS

SEQ ID NO: 15 - protein, 112 aa, synthetic construct
DIQMTQSPSSLSASVGDRVTITCRSSQSIVHNDGNTYFEWYQQKPGKAPKLLIYKVSNRFSGVPSRFSGSGSGTHFTLTISSLQPEDFATYYCFQGSYVPLTFGQGTKVEIK

SEQ ID NO: 16 - protein, 120 aa, synthetic construct
EVQLVESGGGLVQPGGSLRLSCAASGFTFSSFGMHWVRQAPGKGLEWVAYINGGSSTIFYANAVKGRFTISRDNAKNTLYLQMNSLRAEDTAVYYCARYASYGGGAMDYWGQGTLVTVSS

SEQ ID NO: 17 - protein, 219 aa, synthetic construct
DIQMTQSPSSLSASVGDRVTITCRSSQSIVHNDGNTYFEWYQQKPGKAPKLLIYKVSNRFSGVPSRFSGSGSGTHFTLTISSLQPEDFATYYCFQGSYVPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 453 aa, synthetic construct [sequence omitted]

SEQ ID NO: 19 - protein, 137 aa, synthetic construct
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 20 - protein, 573 aa, synthetic construct
EVQLVESGGGLVQPGGSLRLSCAASGFTFSSFGMHWVRQAPGKGLEWVAYINGGSSTIFYANAVKGRFTISRDNAKNTLYLQMNSLRAEDTAVYYCARYASYGGGAMDYWGQGTLVTVSSGSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 21 - protein, 412 aa, Homo sapiens
MPLQLLLLLILLGPGNSLQLWDTWADEAEKALGPLLARDRRQATEYEYLDYDFLPETEPPEMLRNSTDTTPLTGPGTPESTTVEPAARRSTGLDAGGAVTELTTELANMGNLSTDSAAMEIQTTQPAATEAQTTQPVPTEAQTTPLAATEAQTTRLTATEAQTTPLAATEAQTTPPAATEAQTTQPTGLEAQTTAPAAMEAQTTAPAAMEAQTTPPAAMEAQTTQTTAMEAQTTAPEATEAQTTQPTATEAQTTPLAAMEALSTEPSATEALSMEPTTKRGLFIPFSVSSVTHKGIPMAASNLSVNYPVGAPDHISVKQCLLAILILALVATIFFVCTVVLAVRLSRKGHMYPVRNYSPTEMVCISSLLPDGGEGPSATANGGLSKAKSPGLTPEPREDREGDDLTLHSFLP

SEQ ID NO: 22 - protein, 402 aa, synthetic construct
MPLQLLLLLILLGPGNSLQLWDTWADEAEKALGPLLARDRRQATEYEYLDYDFLPETEPPEMLRNSTDTTPLTGPGTPESTTVEPAARRSTGLDAGGAVTELTTELANMGNLSTDSAAMEIQTTQPAATEAQTTPLAATEAQTTRLTATEAQTTPLAATEAQTTPPAATEAQTTQPTGLEAQTTAPAAMEAQTTAPAAMEAQTTPPAAMEAQTTQTTAMEAQTTAPEATEAQTTQPTATEAQTTPLAAMEALSTEPSATEALSMEPTTKRGLFIPFSVSSVTHKGIPMAASNLSVNYPVGAPDHISVKQCLLAILILALVATIFFVCTVVLAVRLSRKGHMYPVRNYSPTEMVCISSLLPDGGEGPSATANGGLSKAKSPGLTPEPREDREGDDLTLHSFLP

SEQ ID NO: 23 - protein, 219 aa, synthetic construct; identical to SEQ ID NO: 17

SEQ ID NO: 24 - protein, 447 aa, synthetic construct
EVQLVESGGGLVQPGGSLRLSCAASGFTFSSFGMHWVRQAPGKGLEWVAYINGGSSTIFYANAVKGRFTISRDNAKNTLYLQMNSLRAEDTAVYYCARYASYGGGAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK